(19) United States
(12) Patent Application Publication
(10) Pub. No.: US 2021/0371940 A1
(43) Pub. Date: Dec. 2, 2021

(54) PROCESS FOR IDENTIFYING AND QUANTIFYING NUCLEIC ACID SEQUENCES FROM LIVE MICROORGANISMS

(71) Applicant: Cicadea Biotech, LLC, St. Louis, MO (US)

(72) Inventor: WEN CHEN BURNS, CHESTERFIELD, MO (US)

(21) Appl. No.: 17/331,460

(22) Filed: May 26, 2021

Related U.S. Application Data
(60) Provisional application No. 63/031,350, filed on May 28, 2020.

(51) Int. Cl. C12Q 1/6895; C12Q 1/689; C12Q 1/6893

(52) U.S. Cl. CPC C12Q 1/6895; C12Q 2600/158; C12Q 1/6893; C12Q 1/689

(57) ABSTRACT

Described are methods for isolating, amplifying and analyzing nucleic acids from live cells in mixed cultures suspected of containing living cells, dead cells, and/or free nucleic acids released extracellularly from dead or dying cells. Also provided are processed test samples and kits for preparation thereof.

### RELATED APPLICATIONS

This application claims priority to U.S. Provisional Application No. 63/031,350 filed May 28, 2020, the entire disclosure of which is hereby incorporated by reference.

### INCORPORATION BY REFERENCE OF SEQUENCE LISTING

This application contains a Sequence Listing that has been submitted in ASCII format via EFS-Web and is hereby incorporated by reference in its entirety. The ASCII copy, created on May 18, 2021, is named “111315-684924_ST25.txt” and is 4.6 kilobytes in size.

### FIELD

The present disclosure is generally directed to methods for isolating, amplifying and analyzing nucleic acids from live cells in mixed cultures suspected of containing living cells, dead cells, and/or free nucleic acids released extracellularly from dead or dying cells.

### BACKGROUND

An ongoing need exists for identification and quantification of microorganisms in environmental, clinical, consumable (e.g., food) and investigative samples. There is also a need to distinguish living from dead microorganisms. Some examples of microorganisms that need to be identified or quantified in clinical or environmental samples include eukaryotic microorganisms such as protists, yeasts, and fungi, and prokaryotic microorganisms, such as Gram negative and Gram-positive bacteria, as well as viral agents.

Polymerase Chain Reaction (PCR) methods can be used to identify, quantify, and analyze both prokaryotic and eukaryotic microorganisms. However, current PCR methods cannot distinguish between live and dead microorganisms or free nucleic acids. This is because nucleic acids can persist in an environment even after the source organism has died (e.g., within the dead organism or as released free or extracellular nucleic acid). This background level of nucleic acids can interfere with a viability determination of a microorganism sample or culture. Accordingly, free/extracellular nucleic acids and those from dead cells can be considered contaminants when measuring levels of living organisms/cells in a sample using a PCR based method. Successful viability testing of microbial cultures thus requires minimizing detection of nucleic acids from dead cells or in the extracellular environment. Certain compounds such as ethidium monoazide (EMA) and propidium monoazide (PMA) have been used in combination with PCR to detect live microorganisms. However, these compounds can form complexes with free DNA that can interfere with PCR amplifications. Further, methods using these compounds are fine for small scale laboratory research and development purposes, but require complicated procedures and technical expertise, including the use of a darkroom and a special light transmitter, and can be quite costly and laborious. As such, these methods are currently not practical or scalable for use in commercial applications.

### BRIEF SUMMARY

In accordance with an aspect of the disclosure, a screening method is provided for detecting the presence of one or more live cells of interest in a test sample suspected of comprising the live cells of interest and at least one dead cell or free nucleic acid material, the method comprising: (a) obtaining or having obtained a retentate formed by filtering the test sample through a filter, wherein the retentate comprises live cells present in the test sample; b) lysing the live cells in the retentate to form a lysed cell mixture comprising nucleic acid from the lysed cells; (c) filtering the lysed cell mixture from (b) through the filter to form a filtrate comprising the nucleic acid from the lysed cells; (d) subjecting the filtrate from (c) to a polymerase chain reaction (PCR) to amplify the nucleic acid from the lysed cells; and (e) analyzing the amplified nucleic acid to determine whether the live cells of interest are present in the test sample.

In another aspect of the disclosure, a method for correcting or compensating for the presence of dead cells and free nucleic acid from dead cells in a test sample to be subjected to PCR amplification for detecting the presence of live cells of interest in the test sample is provided. The method can comprise (a) filtering the test sample to form a retentate comprising the live cells and a first filtrate comprising free nucleic acid, dead cell material, or a combination thereof; (b) lysing the live cells in the retentate to form a lysed cell mixture comprising nucleic acid from the lysed cells; (c) filtering the lysed cell mixture from (b) through the filter to form a filtrate comprising the nucleic acid from the lysed cells; (d) subjecting the filtrate from (c) to polymerase chain reaction (PCR) to amplify the nucleic acid from the lysed cells; (e) analyzing the amplified nucleic acid to detect the presence of the live cells of interest present in the test sample.

In various aspects of the disclosure, the screening method provided further comprises filtering the test sample to form a retentate comprising the live cells and a first filtrate comprising free nucleic acid, dead cell material or a combination thereof.

In various aspects of the disclosure, the one or more live cells suspected of being present in the test sample can comprise one or more microorganisms. The one or more live cells suspected of being present in the test sample can comprise one or more of bacteria, archaea, fungi, yeasts, molds, algae, protists, or protozoa.

In further aspects of the disclosure, the filter does not bind or poorly binds free nucleic acids such that a filtrate formed according to the methods herein comprises a majority of free nucleic acid present in the test sample. In various aspects of the disclosure, the filter has pores having a diameter of less than 0.5, less than 0.4, less than 0.3 or less than 0.2 microns.

In various aspects of the methods provided herein, the test sample can comprise a fluid test sample or a solid or semi-solid prepared as a fluid suspension or solution.

In various aspects of the disclosure, the methods provided herein are free of an added nucleic acid binding agent for extracting free nucleic acid from the test sample.

In various aspects of the disclosure, the test sample comprises waste water, household water, municipal water, irrigation water, water obtained from a water tank or water reservoir, swimming pool water, bodies of water, building coolant fluid, building heater fluid, environmental water and fluids, fluid or solids from microbial fermentations, human or animal bodily tissue, solids, or fluid, human waste products and materials, food or drink, animal feed, pet food, medical supplies, swab or survey collections from solid surfaces, medical waste, human and animal drugs, nutraceuticals, probiotic or prebiotic supplements, plant materials, soil, agriculture production inputs, harvested agriculture commodities or produce, gaseous material, or a fluid sample prepared therefrom. In further aspects, the test sample that comprises food or drink can comprise a feed, milk, meat, fermented products, beer, wine, cheese, soy sauce, fish or shellfish, seafood, poultry, fruit, vegetables or eggs or products made therefrom.

In further aspects of the disclosure, lysing the cells comprises agitating the live cells in the presence of beads, granules, pellets or other solid material suitable for disrupting cell membranes. In various aspects, agitating the live cells can comprise sonication, magnetic agitation, use of a forced gas of any combination thereof.

In still further aspects, filtering the test sample and/or filtering the lysed cell mixture further comprises applying a centrifugal or vacuum force across the filter.

In various aspects, the methods provided herein comprise a polymerase chain reaction (PCR) comprising Real-Time PCR (quantitative PCR or qPCR), Reverse-Transcriptase (RT-PCR), Multiplex PCR, Nested PCR, High Fidelity PCR, Fast PCR, Hot Start PCR, Long-range PCR, Arbitrary Primed PCR, Digital PCR, Droplet Digital PCR (ddPCR), isothermal amplification PCR, or Endpoint PCR (Qualitative PCR). In various aspects, the polymerase chain reaction comprises qPCR or ddPCR.

In various aspects, analyzing the amplified nucleic acid in step (e) comprises sequencing, quantitating, or qualitatively detecting the amplified nucleic acid.

In various aspects, sequencing the amplified nucleic acid comprises Sanger sequencing, single molecule real time (SMRT) sequencing, nanopore sequencing, massively parallel signature sequencing (MPSS), colony sequencing, 454 pyrosequencing, illumina sequencing, combinatorial probe anchor synthesis (cPAS), SOLiD sequencing, ion torrent semiconductor sequencing, nanoball sequencing, heliscope single molecule sequencing, using a microfluidic system or a combination of any thereof.

In further aspects, quantitating the amplified nucleic acid can occur in any quantitative polymerase chain reaction described above (e.g., qPCR). In various aspects, quantitating the amplified nucleic acid can comprise use of nanotechnology (e.g., a nanopore). In additional aspects, quantitating the amplified nucleic acid can comprise use of an oligonucleotide probe or nucleic acid dye.

In still further aspects, the amplified nucleic acid can be qualitatively detected using, for example, an agarose gel, polyacrylamide electrophoresis, restriction endonuclease digestion, dot blots, liquid chromatography, electrochemoluminescence, or a combination of any thereof.

In various aspects, the live cells suspected of being present in the test sample are pathogenic or indicative of the presence of pathogenic live cells. In various aspects, the live cells suspected of being present in the test sample are toxin producing cells or indicative of the presence of toxin producing cells. In further aspects, the live cells suspected of being present in the test sample comprise one or more bacteria, archaea, fungi, yeasts, molds, algae, protists, or protozoa. In various aspects, the protist or protozoan is an amoeba, cryptosporidium, or Giardia. In still further aspects, the microorganism is a mycoplasma, or a bacterium which is a Gram-negative bacterium, or a Gram-positive bacterium. The bacterium can be selected from any one of the genera Legionella, Escherichia, Salmonella, Listeria, Candidatus, Vibrio, Proteus, Pseudomonas, Borrelia, Mycobacterium, Bacillus, Klebsiella, Yersinia, Enterococcus, Aerococcus, Brucella, Shigella, Acinetobacter, Clostridium, Staphylococcus, Enterococcus, Pseudomonas, Acinetobacter, Streptococcus, Klebsiella, or is a fecal coliform bacterium.

In accordance with another aspect of the disclosure, a processed test sample is provided. The processed test sample can be prepared from an original test sample for PCR amplification of nucleic acid from live cells of interest suspected of being present in the original test sample, the processed test sample comprising nucleic acid from the live cells substantially free of free nucleic acid and/or dead cells present in the original test sample.

In various aspects, the processed test sample provided herein can be obtained by a process comprising a first filtering of the original test sample through a filter to form a retentate, wherein the retentate comprises a majority of the live cells from the test sample; lysing the live cells in the retentate to form a lysed cell mixture comprising nucleic acid from the lysed cells; and filtering the lysed cell mixture through the filter to form a filtrate comprising the nucleic acid from the lysed cells.

In various aspects, the processed test sample is free of an added nucleic acid binding agent for extracting free nucleic acid from the test sample.

In further aspects, the processed test sample comprises nucleic acid from one or more live microorganisms present in the original sample.

In still further aspects, the live cells of interest in the processed test sample are pathogenic or indicative of the presence of pathogenic live cells. In various aspects, the live cells of interest are toxin producing cells or indicative of the presence of toxin producing cells. In various aspects, the live cells of interest comprise one or more microorganisms selected from bacteria, archaea, fungi, yeasts, molds, algae, protists, or protozoa. In further aspects, the microorganisms comprise one or more of a mycoplasma, a Gram negative bacterium, or a Gram positive bacterium. In still further aspects, the bacteria can be selected from the genera Legionella, Escherichia, Salmonella, Listeria, Candidatus, Vibrio, Proteus, Pseudomonas, Borrelia, Mycobacterium, Bacillus, Klebsiella, Yersinia, Enterococcus, Aerococcus, Brucella, Shigella, Acinetobacter, Clostridium, Staphylococcus, Enterococcus, Pseudomonas, Acinetobacter, Streptococcus, Klebsiella, or is a fecal coliform bacterium.

In various aspects, the processed test samples are prepared using a filter that does not bind nucleic acids or poorly binds nucleic acids such that a filtrate formed from filtering the original test sample through the filter comprises a majority of free nucleic acid present in the original test sample. In various aspects, the filter has pores having a diameter of less than 0.5, less than 0.4, less than 0.3 or less than 0.2 microns.

In various aspects, the processed test samples are prepared from an original test sample comprising a fluid test sample or a solid or a semisolid prepared as a fluid suspension or solution. In various aspects, the original test sample used to prepare the processed test sample comprises waste water, household water, municipal water, irrigation water, water obtained from a water tank or water reservoir, swimming pool water, building coolant fluid, building heater fluid, environmental water and fluids, fluid from microbial fermentations, human or animal bodily fluid, fluid obtained from food or drink, plant materials, soil, gaseous material, or a fluid sample prepared therefrom. In various aspects, the original test sample used to prepare the processed test sample comprises food or drink (e.g., a feed, milk, meat, fish and shellfish, seafood, poultry, fruit, vegetables, or eggs).

In various aspects, in preparing the processed test samples, lysing the cells comprises agitating the live cells in the presence of beads, granules, pellets or other solid material suitable for disrupting cell membranes. In various aspects, agitating the live cells can comprise sonication, magnetic agitation, use of a forced gas of any combination thereof.

In further aspects, the processed test samples are prepared by methods comprising filtering the original test sample and/or filtering the lysed cell mixture by applying a centrifugal or vacuum force across the filter.

In various aspects, a kit is provided for preparing a processed test sample described herein. In various aspects, the kit comprises a filter and a container for collecting the processed test sample. In further aspects, the filter in the kit comprises a filter plate or a filter tube. In various aspects, the filter can comprise pores having a diameter of less than 0.5, less than 0.4, less than 0.3 or less than 0.2 microns. In still further aspects, the kit can also comprise a buffer, a nucleic acid probe, an oligonucleotide primer, an enzyme, a substrate, a gel, a nucleic acid dye, a microarray, or a combination of any thereof.

### BRIEF DESCRIPTION OF THE DRAWINGS

FIG. 1 is a diagram of a process for isolating DNA from live microorganisms for viability testing.

FIG. 2 is a diagram comparing a standard Legionella PCR testing process with an exemplary novel viability assay.

FIG. 3 is a diagram of a viability testing process according to certain aspects of the present disclosure.

FIG. 4 is a diagram of a chemical viability testing process.

FIG. 5 is a dilution curve for samples prepared according to certain aspects of the present disclosure. Dilution group shown on the X axis are: 1-10¹ cells/μl, 2-10² cells/μl, 3=10³ cells/μl, 4=10⁴ cells/μl, and 5=10⁵ cells/μl.

FIG. 6 is a dilution curve comparison for samples prepared as described herein in the presence or absence of a nucleic acid binding agent (palladium acetate, PD).

FIG. 7 is a dilution curve of samples amplified using PCR after increasing numbers of rinsing steps.

FIG. 8 is a plot showing PCR amplification of dead cell DNA remaining following sequential rinsing steps (0, 1, 2, or 3 rinses) of samples having different starting levels of dead cells.

FIG. 9 is a diagram showing a PCR based viability testing protocol using a filter tube.

FIG. 10 is a plot showing PCR amplification of dead cell DNA following sequential rinsing steps. Lines represent different samples having different proportions of living and dead cells.

FIG. 11 is a plot depicting levels of DNA extracted from various mixed samples in FIG. 15.

FIG. 12 is a dilution curve from mixed samples analyzed using a cell culture method or a PCR method.

FIG. 13 is a dilution curve comparison from live cell samples analyzed using a cell culture method or a PCR method.

FIG. 14 is a dilution curve comparison from live cell samples analyzed using a cell culture method or a PCR method.

FIG. 15 is a dilution curve comparison of live or mixed samples analyzed using a cell culture method or a PCR method.

FIG. 16 is a dilution curve comparison of live cell samples following rinsing with palladium acetate (PD) or H₂O.

FIG. 17 is dilution curve of live cell samples (prepared without rinsing) or mixed cell samples following sequential rinsing steps.

FIG. 18 is a dilution curve comparison between mixed cell samples rinsed with palladium acetate (PD) and H₂O and live samples with no rinsing.

FIG. 19 is a dilution curve comparison between mixed cell samples rinsed twice with H₂O and live samples with no rinsing.

FIG. 20 is a dilution curve comparison between mixed cell samples rinsed twice with palladium acetate (PD) and live samples with no rinsing.

FIG. 21 is a dilution curve from test samples prepared using a filter plate compared with a dilution curve from DNA standards.

FIG. 22 is a dilution curve from test samples prepared using filter tubes compared with a dilution curve from DNA standards.

FIG. 23 is a diagram of three short DNA protocols for viability testing.

FIG. 24 is a diagram of two long DNA protocols for viability testing.

FIG. 25 is a bar graph showing number of viable cells detected using the five different DNA protocols depicted in FIG. 23 and FIG. 24 (S1 to S5).

FIG. 26 is a diagram of four DNA protocols for viability testing.

FIG. 27 is a bar graph showing log number of viable cells detected using the four DNA protocols depicted in FIG. 3A.

FIG. 28 is bar graph showing log number of viable cells using three different DNA extraction protocols using three different volumes of Tris-EDTA buffer (A-8: 200 μL, B-8: 100 μL, and P1-031921: 400 μL).

FIG. 29 is a diagram showing a protocol for screening for viable bacteria in a sample.

FIG. 30 is a dilution curve comparison between DNA extracted using a long protocol from a first Gram positive bacteria.

FIG. 31 is a dilution curve comparison between DNA extracted using a short protocol from a first Gram positive bacteria.

FIG. 32 is a dilution curve comparison between DNA extracted using a long protocol from a second Gram positive bacteria.

FIG. 33 is a dilution curve comparison between DNA extracted using a short protocol from a second Gram positive bacteria.

FIG. 34 is a dilution curve comparison between DNA extracted from a mixed (unfiltered sample) compared to mixed sample with filtration.

FIG. 35 is a dilution curve comparison between DNA extracted using a long protocol from Escherichia bacteria.

FIG. 36 is a dilution curve comparison between DNA extracted using a short protocol from Escherichia bacteria.

FIG. 37 is a dilution curve comparison between filtered samples containing only live cells (L-), filtered mixed samples containing live and dead cells (F-) and unfiltered mixed samples containing live and dead cells (M-). DNA was extracted using a long protocol.

FIG. 38 is a dilution curve comparison between five samples (L: live cells without filtration, LF: live cells with filtration; SEF: mixed samples with filtration; SEH1: mixed samples with filtration and washing step; and SEN: mixed samples without filtration) of Gram-negative bacteria.

FIG. 39 is a dilution curve comparison of three samples (L, SEF and SEN) to show differences between mixed/live cell samples and filtered/unfiltered samples.

FIG. 40 is a qPCR dilution curve comparison of three samples (M: mixed, unfiltered; F: mixed, filtered; and L: live, filtered) of Legionella bacteria prepared using the long protocol.

FIG. 41 is a ddPCR dilution curve comparison of three samples (M: mixed, unfiltered; F: mixed, filtered; and L: live, filtered) of Legionella bacteria prepared using the long protocol.

FIG. 42 is a dilution curve comparison of three samples (M: mixed, unfiltered; F: mixed, filtered; and L: live, filtered) of Legionella bacteria prepared using the long protocol and subjected to a viability screening.

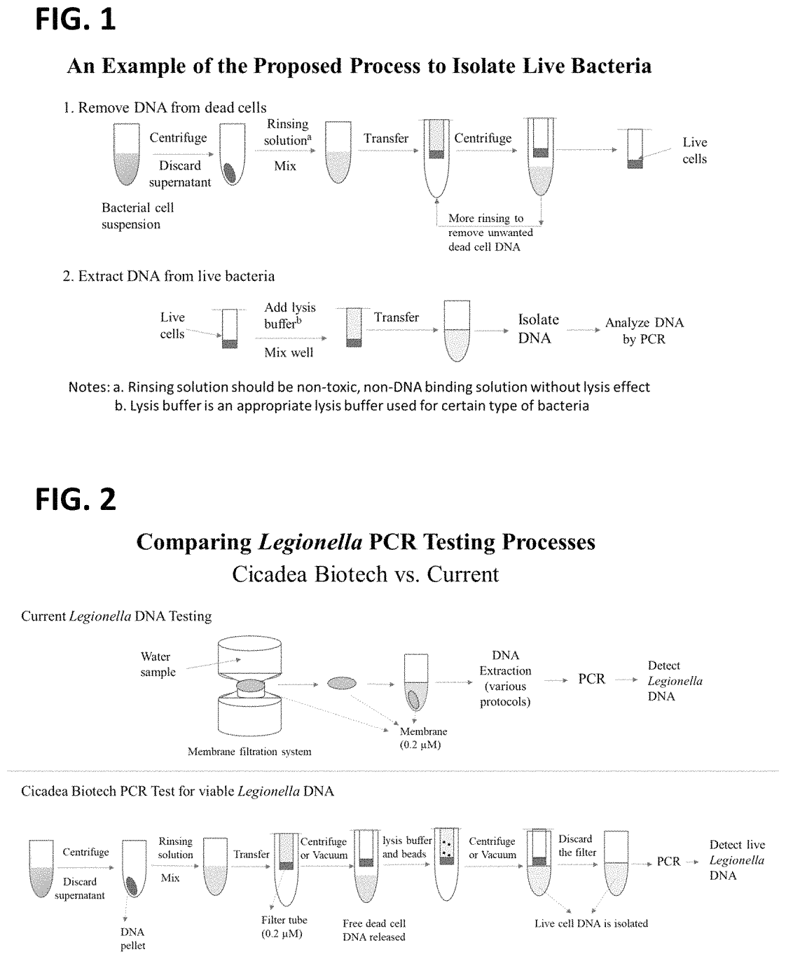

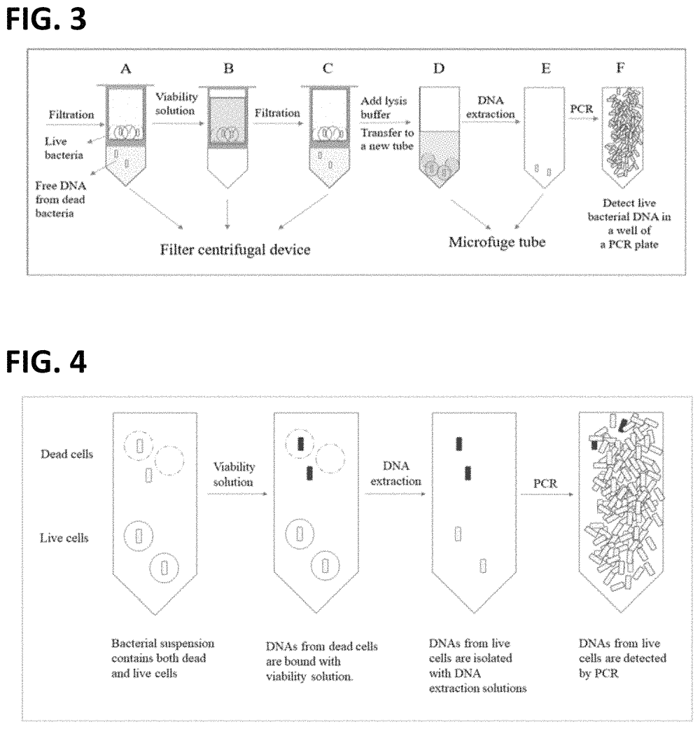

### DETAILED DESCRIPTION

In general aspects, the present disclosure is directed to methods for isolating and amplifying nucleic acids from living organisms in a test sample that may contain living, dead, and/or free (e.g., extracellular) nucleic acids. Accordingly, various screening methods provided herein can be used to detect the presence of one or more live cells of interest and/or correct or compensate for the presence of dead cells and free nucleic acid from dead cells in a test sample suspected of comprising live cells, dead cells, and/or free nucleic acids. The methods described herein exploit a surprising and unexpected finding that live cells can be accurately detected from a mixed sample without the use of DNA binding agents required by many standard protocols. As such they overcome limitations inherent in chemical methods using, for example ethidium monoazide (EMA) and propidium monoazide (PMA), which work poorly with some microorganisms (like Gram positive bacteria) and can interfere with PCR based protocols. Instead, the proposed viability PCR testing strategy in this disclosure is based on physical separation of live cells and/or free DNA from the microorganism prior to PCR detection. This helps to minimize damage to the live cells that may be caused by chemical compounds such as EMA and PMA. As a result, higher accuracy is obtained when using the proposed viability PCR testing strategy. In addition, the proposed strategy is an innovative strategy for high-throughput screening for live microbes.

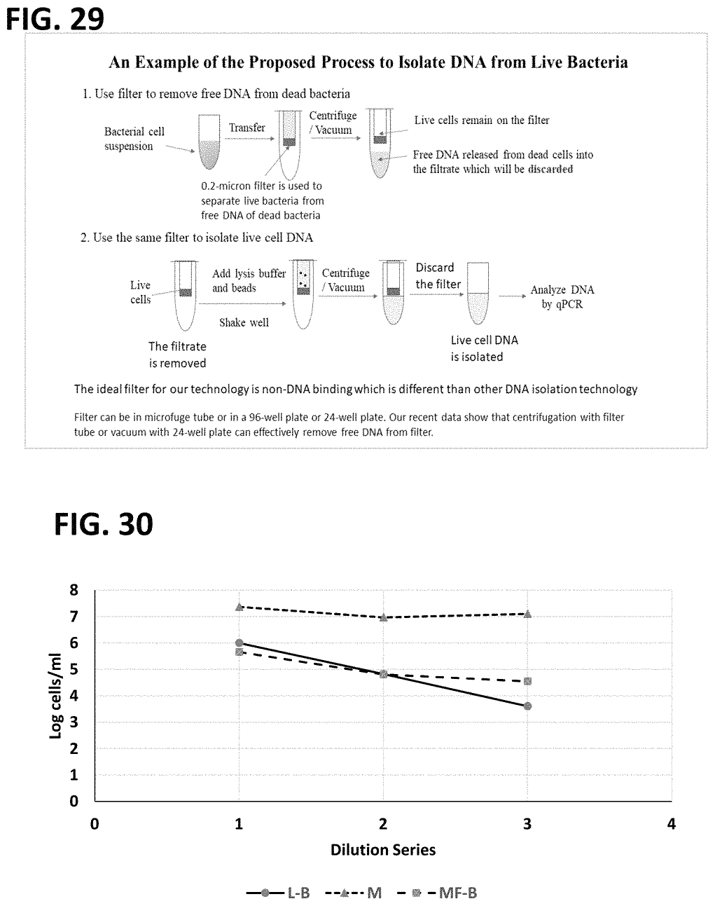

In various aspects, the methods provided herein comprise membrane filtration technology to physically separate live cells from dead cells and free nucleic acids. As shown in FIG. 29, in contrast to standard technology, the methods described herein can use the same filtration device for removal of dead cell DNA and isolation of live cell DNA. The novel methods herein are inexpensive and amenable to many laboratory and experimental conditions, which makes them widely applicable across many applications and industries such as research work and high-throughput screening for live microorganisms.

### I. Methods

In various aspects, the disclosure provides a screening method for detecting the presence of one or more live cells of interest in a test sample suspected of comprising live cells, dead cells, and/or free nucleic acids, the method comprising: (a) obtaining or having obtained a retentate formed by filtering the test sample through a filter, wherein the retentate comprises live cells present in the test sample; (b) lysing the live cells in the retentate to form a lysed cell mixture comprising nucleic acid from the lysed cells; (c) filtering the lysed cell mixture from (b) through the filter to form a filtrate comprising the nucleic acid from the lysed cells; (d) subjecting the filtrate from (c) to a polymerase chain reaction (PCR) to amplify the nucleic acid from the lysed cells; and (e) analyzing the amplified nucleic acid to determine whether the live cells of interest are present in the test sample. In various aspects, the screening method further comprises filtering the test sample to form the retentate comprising the live cells and a first filtrate comprising free nucleic acid, dead cell material, or a combination thereof.

In further aspects, the disclosure also provides a method of correcting or compensating for the presence of dead cells and free nucleic acid from dead cells in a test sample to be subjected to PCR amplification for detecting the presence of live cells of interest in the test sample, the method comprising: (a) filtering the test sample to form a retentate comprising the live cells and a first filtrate comprising free nucleic acid, dead cell material, or a combination thereof; (b) lysing the live cells in the retentate to form a lysed cell mixture comprising nucleic acid from the lysed cells; (c) filtering the lysed cell mixture from (b) through the filter to form a filtrate comprising the nucleic acid from the lysed cells; (d) subjecting the filtrate from (c) to polymerase chain reaction (PCR) to amplify the nucleic acid from the lysed cells; and (e) analyzing the amplified nucleic acid to detect the presence of the live cells of interest present in the test sample.

In various aspects, the methods herein comprise filtering a test sample to form a retentate. Filtering the test sample is used to separate dead cell DNA from living cells. This method is based on the surprising and serendipitous finding that dead cell DNA can be effectively isolated and removed from a sample using filtration technology alone, without the use of DNA binding chemicals (as described in more detail below).

The original test sample can comprise a fluid sample or a solid or a semisolid prepared as a fluid suspension or solution. The original test sample can be obtained from a variety of sources including, but not limited to: waste water, household water, municipal water, irrigation water, water obtained from a water tank or water reservoir, swimming pool water, bodies of water, building coolant fluid, building heater fluid, environmental water and fluids, fluid or solids from microbial fermentations, human or animal bodily tissue, solids, or fluid, human waste products and materials, food or drink, animal feed, pet food, medical supplies, swab or survey collections from solid surfaces, medical waste, human and animal drugs, nutraceuticals, probiotic or prebiotic supplements, plant materials, soil, agriculture production inputs, harvested agriculture commodities or produce, gaseous material, or a fluid sample prepared therefrom. In various embodiments, the test sample comprises plant material or a water sample (e.g., obtained from a public water source or from a waste water source). In various aspects, the test sample comprises food and drink. In various aspects, the food or drink comprises a feed, milk, meat, fermented products, beer, wine, cheese, soy sauce, fish or shellfish, seafood, poultry, fruit, vegetables or eggs or products made therefrom.

In various aspects, filtering the test sample uses a filter that does not bind or poorly binds free nucleic acids. This is different from standard methods where a filter may be treated with a nucleic acid binding agent to remove free nucleic acid and enrich for intact living cells in a sample, or where samples may be further treated with a nucleic acid binding agent to bind and remove free nucleic acids. Accordingly, the filtrate formed in step (a) comprises a majority of free nucleic acid present in the test sample. In various aspects, the filter has pores small enough to retain living cells on the filter but not dead cells or free nucleic acids.

In various aspects, the filter has pores having a diameter of less than 0.5, less than 0.4, less than 0.3, or less than 0.2 microns. In various aspects, the filter has pores having a diameter from 0.1 microns to 0.5 microns. For example, the filter can have pores of about 0.5, about 0.4, about 0.3, about 0.2 microns or about 0.1 microns. In further aspects, the filter has pores having a diameter of 0.5, 0.4, 0.3, 0.2, or 0.1 microns. For example, the filter can have pores having a diameter of about 0.2 microns. In various aspects, the filter can comprise a filter tube. Another non-limiting filter that can be used in the methods provided herein is a filter plate. For example, one non-limiting filter plate that can be used can be a Pall Corporation 96-well filter plate (AcroPrep Advance 1 ml, P8119, 0.2 μM supor). This filter plate can be used for testing up to 96 samples. Furthermore, it can be centrifuged by stacking on top of a 96-well deep well plate.

In further aspects, a retentate formed in step (a), comprising a majority of live cells present in the test sample, is further processed. In some aspects, in a step (b), the live cells in the retentate are lysed to form a lysed cell mixture comprising nucleic acid from the lysed cells

In various aspects, lysing the live cells comprises agitating the live cells in the presence of beads, granules, pellets or other solid material suitable for disrupting cell membranes. Any bead available to facilitate the lysis of cells can be used. For example, lysing the live cells can comprise using glass beads, chrome beads, steel beads, chrome-steel beads, silicon carbide beads, garnet beads, stainless steel beads, granules, pellets, plastic beads, ceramic beads, or a combination of any thereof. The agitation can comprise mechanical agitation methods including, but not limited to, sonication, magnetic, forced gas, etc. In various embodiments, the beads are glass beads. The beads can be of any size conventionally used in the art. For example, the beads can be from about 1 to 5 mm or from about 2 to 3 mm.

In various aspects, lysing the live cells comprises applying a lysis buffer to the cells. Suitable lysis buffers can include, but are not limited to, SDS buffers or Tris-EDTA (TE) buffers. In various aspects the SDS buffer can comprise Tris-HCl, EDTA, KCl, and SDS. In various aspects, the TE buffer can comprise Tris-HCl and EDTA. For example, the SDS buffer can comprise about 200 mM Tris-HCl, about 20 mM EDTA, about 1 M KCl, about 1% SDS and the TE can comprise about 10 mM Tris-HCl and about 1 mM EDTA.

In various aspects, lysing the cells can comprise heating the cells to an elevated temperature (e.g., above 90° C., above 95° C., or above 99° C.).

In various aspects, the lysed cell mixture is filtered a second time to separate free DNA of interest from the lysed cells. Preferably, the same filter used to form the filtrate in step (a) is used in step (c). This minimizes loss of cells and/or nucleic acids by transferring to a different system and increases the precision and accuracy of the method.

In various aspects, filtering the test sample (e.g., in step (a)) and/or filtering the lysed cell mixture (e.g., in step (c)), further comprises applying a centrifugal or vacuum force across the filter. One non-limiting type of filter centrifugal device that can be used in methods of the present teachings is a Pall Corporation 0.2 micron Nanosep centrifuge tube (ODM02C33) which is composed of a 0.2 micron filter insert and a 1.5 ml microfuge tube, a Pall Corporation AcroPrep 24-well sterile filtration plate-0.65/0.2 μm Supor EKV, and a AcroPrep Advance 1 ml, PN 8119, 0.2 uM Supor (96-well filter plate).

In various aspects, the methods provided herein can further comprise a rinsing step wherein the microfuge tube or plate is rinsed with a suitable solution to facilitate removal of residual free or dead DNA. In various embodiments, the suitable solution comprises water. In some embodiments, the suitable solution comprises a buffer (e.g., a neutral and cell-stable buffer).

As noted, the methods provided herein can comprise subjecting the filtrate formed in step (c) to a polymerase chain reaction (PCR) to amplify the nucleic acid from the lysed cells. Any polymerase chain reaction known in the art can be used to amplify the nucleic acids. In various aspects, the polymerase chain reaction comprises Real-Time PCR (quantitative PCR or qPCR), Reverse-Transcriptase (RT-PCR), Multiplex PCR, Nested PCR, High Fidelity PCR, Fast PCR, Hot Start PCR, Long-range PCR, Arbitrary Primed PCR, Digital PCR, Droplet Digital PCR (ddPCR), isothermal amplification PCR, Endpoint PCR (Qualitative PCR), or a combination of any thereof. In various aspects, the polymerase chain reaction comprises qPCR or ddPCR.

Each of the PCR reactions described above can be performed according to standard methods in the art, including those provided in laboratory manuals such as Sambrook, J., et al. Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; Carruthers, W., and Coldham, I., Modern Methods of Organic Synthesis (4th Edition), Cambridge University Press, Cambridge, U.K., 2004, herein incorporated by reference in its entirety.

As noted, the methods provided herein comprise analyzing an amplified nucleic acid produced by a PCR protocol. In various aspects, the analysis can comprise sequencing the nucleic acid using, but not limited to, Sanger sequencing, single molecule real time (SMRT) sequencing, nanopore DNA sequencing, massively parallel signature sequencing (MPSS), colony sequencing, 454 pyrosequencing, illumina sequencing, combinatorial probe anchor synthesis (cPAS), SOLiD sequencing, ion torrent semiconductor sequencing, DNA nanoball sequencing, heliscope single molecule sequencing, using a microfluidic system or a combination of any thereof.

In other aspects, the analysis can comprise quantitative detection of the nucleic acid, such as using an oligonucleotide probe or nucleic acid dye. In various aspects, the quantitative detection occurs as part of the polymerase chain reaction performed (e.g., using real time PCR or quantitative PCR (qPCR). In other aspects, the quantitative detection occurs after the polymerase chain reaction is performed. For example, in some aspects, the quantitation can use a nanopore or similar nanotechnology to detect the nucleic acids. See for example Kang et al., “Ready-to-use nanopore platform for the detection of any DNA/RNA oligo at attomole range using an Osmium tagged complementary probe” Scientific Reports 10, 19790 (2020), which is incorporated herein by reference in its entirety. In still further aspects, the analysis can comprise qualitative detection, such as using an agarose gel, polyacrylamide electrophoresis, restriction endonuclease digestion, dot blots, liquid chromatography, electrochemoluminescence, or a combination of any thereof. In general, any method known in the art to sequence, quantify, or detect the nucleic acid may be used in the methods provided herein. In any aspect provided herein, identifying the nucleic acid as indicative of the presence of the live cell of interest is contemplated.

In various aspects, the methods provided herein are suitable for detecting certain microorganisms in a sample. Advantageously, the methods provided herein can aid in the selective detection of live microorganisms that are difficult to identify using standard protocols in typical environmental samples.

As a non-limiting example, Legionella are Gram-negative, non-spore-forming, rod-shaped, aerobic bacteria which can contaminate water supplies. Legionnaires' disease (LD) is a severe pneumonia believed to be caused by Legionella bacteria. The current standard testing process for Legionella bacteria is very time consuming due to the use of a culture method, and suffers from poor sensitivity and difficulty culturing. The culture testing method uses a special culture medium. Performing the test requires extensive technical expertise, and 7-14 days are needed to produce results.

Furthermore, plate count data for Legionella using standard laboratory culture methods are not reliable and are prone to false low cell counts compared to data generated from PCR detection methods. The poor quality of data from the culture method is also due to the fact that Legionella are not ordinarily free-living and stay within amoeba in water in a viable but not culturable state (VBNC). Development of rapid, sensitive, cost-effective, and easy-to-operate methods for detection of the VBNC state of Legionella bacteria is an urgent need.

Many Legionella detection platforms have been developed to test different types of samples. These platforms can have poor detection limits and sensitivities. Commercial PCR-based Legionella detection kits do not limit detection to live Legionella and thus can produce false positives or overestimation due to killed bacteria in samples.

As shown in FIG. 2, in an exemplary process described herein, there is no need to transfer the filter membrane to avoid unintentional cross-contamination or unintentional loss of live cells during the transfer or; a sample solution can be concentrated for contaminants (such as Legionella) in the same device used for viability treatment before PCR analysis. As a result, the processes described herein can ensure PCR detection of live bacteria (such as Legionella) and can be used, in various configurations, to serve as valuable surveillance tools for the early detection of the Legionella bacteria in the environment such as water sources, such as showers, hot tubs, water misters, and cooling towers. This process is also very adaptable for high-throughput screening for viable bacteria like Legionella.

As another non-limiting example, Candidatus liberibacter is a Gram-negative bacterium that causes Huanglongbing (HLB) or citrus greening disease which is the most devastating disease impacting citrus production. There is no culture method established for the bacteria. Therefore, PCR-based detection methods have played an important role in detecting the bacteria. It has been reported that the bacteria can stay in a host such as tree stock for three years before symptoms appear. This pathogen can be transmitted by insects such as psyllids. Although PMA PCR has been investigated, inconsistent data was obtained from the only study performed when testing 18 citrus varieties or species and other citrus relatives or other plant species. Therefore, no viability PCR has yet been reported for the detection of viable Candidatus Liberibacter. In various aspects, the methods provided herein can be used to detect live Candidatus Liberibacter in a variety of samples and may also be useful in testing the antimicrobial agents against Candidadus Liberibacter.

Additionally, in any of the methods or compositions described herein, the one or more live cells suspected of being present in the test sample can comprise one or more microorganisms. For example, the live cells can comprise one or more of bacteria, archaea, fungi, yeasts, molds, algae, protists or protozoa. In various aspects, the live cells suspected of being present in the test sample are pathogenic or indicative of the presence of pathogenic live cells. In various aspects, the live cells suspected of being present in the test sample are toxin producing cells or indicative of the presence of toxin producing cells. In various aspects, the protist or protozoan is an amoeba, cryptosporidium, or Giardia. In various aspects, the microorganism can be a mycoplasma, a Gram-negative bacterium, or a Gram-positive bacterium. For example, the bacterium can be any species selected from any one of the genera Legionella, Candidatus, Escherichia (e.g., E. coli), Salmonella, Listeria, Clostridium, Vibrio, Proteus, Pseudomonas, Borrelia, Mycobacterium, Bacillus, Klebsiella, Yersinia, Aerooccus, Brucella, Shigella, Staphylococcus, Enterococcus, or is a fecal coliform bacterium.

Accordingly, in order to detect these and other microorganisms of interest in a test sample, suitable probes and primers appropriate for the given microorganism can be used. Non-limiting examples of PCR primers and probes that can be used in various configurations of the present teachings are provided in Table 1.

TABLE 1
Illustrative primers and probes.

| | SEQ | | |
|---|---|---|---|
| Sequence | ID NO. | Target pathogen | Primer/probe |
| CGCCACTCATAGCGTCTTGC | 1 | Legionella | Probe (6-FAM/BHQ-1) |
| | | pneumophila | |
| GGCATAGATGTTAATCCG | 2 | Legionella | Forward primer |
| | | pneumophila | |
| TTCGGTTAAAGCCAATTG | 3 | Legionella | Reverse primer |
| | | pneumophila | |
| TGCACCAATAAGGCCGCCAA | 4 | Staphylococcus | Probe (6-FAM/BHQ-1) |
| | | aureus | |
| CTGGTGATGATACAGGAA | 5 | Staphylococcus | Forward primer |
| | | aureus | |
| GGTTGAACATATTTCAGTGTA | 6 | Staphylococcus | Reverse primer |
| | | aureus | |
| TCTTCCTGCGATGCCTCACA | 7 | Salmonella enterica | Probe (6-FAM/BHQ-1) |
| CAGCCATTCCTCAAGTAA | 8 | Salmonella enterica | Forward primer |
| CCAGCATACCGTTCTTAA | 9 | Salmonella enterica | Reverse primer |
| CCGATCCGAGCGATGGCATA | 10 | Pseudomonas | Probe (6-FAM/BHQ-1) |
| | | aeruginosa | |
| GGAACTGGAATGGTACTC | 11 | Pseudomonas | Forward primer |
| | | aeruginosa | |
| GGATCGTTGGAGATAGTC | 12 | Pseudomonas | Reverse primer |
| | | aeruginosa | |
| CGCCTGCAAGTCCTAAGACGCCA | 13 | Listeria | Probe (6-FAM/BHQ-1) |
| | | monocytogenes | |
| TTTCATCCATGGCACCACC | 14 | Listeria | Forward primer |
| | | monocytogenes | |
| ATCCGCGTGTTTCTTTTCGA | 15 | Listeria | Reverse primer |
| | | monocytogenes | |
| CCAATTCCTCTGCCCGCTTT | 16 | Enterococcus | Probe (6-FAM/BHQ-1) |
| | | faecalis | |
| CTGGATGATATTTACTATTTATCG | 17 | Enterococcus | Forward primer |
| | | faecalis | |
| GTCAGAGATTCTTTGACTAAA | 18 | Enterococcus | Reverse primer |
| | | faecalis | |
| ACAGTCATCCCCGTAATTTGCG | 19 | Escherichia coli | Probe (6-FAM/BHQ-1) |
| | | O157:H7 | |
| CCAACAGATGGAATCTTCA | 20 | Escherichia coli | Forward primer |
| | | O157:H7 | |
| CTCCATTATGACAGGCATTA | 21 | Escherichia coli | Reverse primer |
| | | O157:H7 | |

### II. Processed Test Samples

A further aspect of the present disclosure provides processed test samples prepared from an original test sample for PCR amplification of nucleic acid from live cells of interest suspected of being present in the original test sample. The processed test sample herein comprises the nucleic acid from the live cells and is substantially free of free nucleic acid and/or dead cells present in the original sample.

The processed test sample can be prepared using any methods described above, starting from an original test sample described above suspected of containing living and dead cells and free nucleic acids. In various aspects, the processed test sample is obtained by a process comprising a first filtering of the original test sample through a filter to form a retentate, wherein the retentate comprises a majority of the live cells from the test sample; lysing the live cells in the retentate to form a lysed cell mixture comprising nucleic acid from the lysed cells; and filtering the lysed cell mixture through the filter to form a filtrate comprising the nucleic acid from the lysed cells. In various aspects, the processed test sample is free of any added nucleic acid binding agent for extracting nucleic acid from the test sample.

In various aspects, the processed test sample comprises nucleic acid from one or more live cells (e.g., microorganisms) present in the original sample. In various aspects, the live cells of interest are pathogenic or indicative of the presence of pathogenic live cells. In various aspects, the live cells of interest are toxin producing cells or indicative of the presence of toxin producing cells. In various aspects, the live cells comprise one or more microorganisms. For example, the live cells can comprise one or more microorganism selected from bacteria, archaea, fungi, yeasts, molds, algae, protists, or protozoa. In various aspects, the protist or protozoan is an amoeba, cryptosporidium, or Giardia. In various aspects, the microorganism can be a mycoplasma, a Gram-negative bacterium, or a Gram-positive bacterium. For example, the bacterium can be any species selected from any one of the genera Legionella, Escherichia (e.g., E. coli), Salmonella, Listeria, Clostridium, Vibrio, Proteus, Pseudomonas, Borrelia, Mycobacterium, Bacillus, Klebsiella, Yersinia, Aerooccus, Brucella, Shigella, Candidatus, Staphylococcus, Enterococcus, or is a fecal coliform bacterium. In various aspects, the bacterium can comprise a bacterium of the genus Legionella, or Candidatus.

In various aspects, lysing the cells comprises agitating the live cells in the presence of beads, granules, pellets or other solid material suitable for disrupting cell membranes. Any bead available to facilitate the lysis of cells can be used. For example, lysing the live cells can comprise using glass beads, chrome beads, steel beads, chrome-steel beads, silicon carbide beads, garnet beads, stainless steel beads, granules, pellets, plastic beads, ceramic beads, or a combination of any thereof. The agitation can comprise mechanical agitation methods including, but not limited to, sonication, magnetic, forced gas, etc. In various embodiments, the beads are glass beads. The beads can be of any size conventionally used in the art. For example, the beads can be from about 1 to 5 mm or from about 2 to 3 mm.

In various aspects, the filter used to obtain the processed test sample does not bind nucleic acids or poorly binds nucleic acids such that a filtrate formed from filtering the original test sample through the filter comprises a majority of free nucleic acid present in the original test sample. In various aspects, the filter has pores having a diameter of less than 0.5, less than 0.4, less than 0.3 or less than 0.2 microns.

In various aspects, the processed test sample is prepared from a process further comprising rinsing the filter, filtrate, and/or retentate with a suitable solution (e.g., water).

### III. Kits

A further aspect of the present disclosure provides kits comprising a filter and a container to be used to prepare a processed test sample described herein. In various aspects, the filter comprises a filter plate or a filter tube described above. In further aspects, the filter comprises pores having a diameter of less than 0.5, less than 0.4, less than 0.3, or less than 0.2 microns. Any filter described above can be included in the kits. In various aspects, the kits can be prepared to identify/quantify/sequence or otherwise detect nucleic acid from any desired microorganism in a variety of test samples. Accordingly, the kits can be formulated with reagents and materials useful for performing any of the steps described herein. various aspects, the kit further comprises a buffer such as a lysis buffer. For example, the kit can comprise a SDS buffer or TE buffer as described herein. In further aspects, the kit can further comprise beads for lysing a live cell. The kit can also comprise reagents (e.g., primers, probes, buffers) for conducting a polymerase chain reaction to amplify a nucleic acid of the live cell of interest. In various aspects, the kit can comprise reagents for analyzing an amplified nucleic acid of the live cell. These can include, for example, standard control nucleic acids (e.g., DNAs) for DNA quantification purposes. Accordingly, the kits can further comprise a buffer, a nucleic acid probe, an oligonucleotide primer, an enzyme, a substrate, a gel, a nucleic acid dye, a microarray, a control nucleic acid, or a combination of any thereof.

Additional components may be added to the kits, according to skills of ordinary person in the art, to facilitate additional analysis of isolated nucleic acids obtained by the kit. These components may vary depending on the nature of the test sample and the target microorganisms. For example, a kit may contain reagents for the simplified DNA extraction for clear water samples and reagents for DNA purification after the simple DNA protocol for dirty test samples. In addition to the filtration device, for example, additional components can be added to further concentrate and purify the nucleic acids. For example, the kit can comprise a column.

The kits may further comprise nucleic acid purification reagents, buffers, and the like. The kits provided herein generally include instructions for carrying out the methods detailed herein. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended.

For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such reference is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples

### EXAMPLES

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1: Effect of Palladium Acetate (PD) on PCR Amplification of Free DNA from a Microorganism

This example illustrates the effect of palladium acetate (PD) on PCR amplification of free DNA from a microorganism, using Legionella pneumophila as a representative DNA source.

Palladium acetate (PD) has been used with PCR for detection of viable bacteria of Enterobacteriaceae such as E. coli. No report has been made for the detection of other microorganisms such as live Legionella. In these experiments, free Legionella DNA that had been previously isolated and quantified on a nanodrop photometer was diluted into test and reference materials with these concentrations provided in Legionella cell equivalence units: 10⁵ Cells/μl, 10⁴ Cells/μl, 10³ Cells/μl, 10² Cells/μl, 10¹ Cells/μl. Cells/ml is equivalent to genome-copy/ml. This set of diluted test materials was then used for different treatments.

### Materials and Methods

Test material preparation: 200 μl of each DNA sample (10⁵ Cells/μl, 10⁴ Cells/μl, 10³ Cells/μl, 10² Cells/μl, and 10¹ Cells/μl) was used for the experiment. They were used to prepare the following study test materials. Standard: only used as a reference standard to quantify other DNA samples in the same PCR experiment. These samples were not used in the listed treatment procedure and no PD treatment was used. LP-NT (Legionella pneumophila no PD treatment): Used as a process control by going through the listed treatment procedure without PD treatment. LP-P (Legionella pneumophila-Palladium acetate): Used as PD treated test materials. It contains 1 μM PD after 2 μl of 100 μM PD was added.

Treatment procedure: 200 μl samples (LP-NT and LP-P) were centrifuged at 13,000 rpm for 5 minutes. 180 μl of the supernatant was discarded, and 180 μl sterile H₂O was added to the pellet.

PCR: Five (5) μl of each sample was combined with 5 μl of the PCR reaction mix to bring up to 10 μl per reaction. Each PCR reaction mix contained a set of Legionella pneumophila-specific primers (forward (SEQ ID NO: 2) and reverse (SEQ ID NO: 3)) (1.0 μM each), a Legionella pneumophila specific probe (0.2 μM, SEQ ID NO: 1), and Life Technology Taqman® Universal Mastermix II (1×). These primers and probe have been designed and validated at Cicadea Biotech, and are provided in Table 1 herein. Two replicates per sample were tested by PCR. A set of diluted DNA standards were used as reference standards to quantify samples. The PCR reactions were carried out on the Life Technology QuantStudio 5 with these cycling conditions: 1 cycle of 50° C. for 2 minutes; 1 cycle of 95° C. for 10 minutes; then 40 cycles of 95° C. for 15 seconds and 60° C. for 30 seconds.

### Results

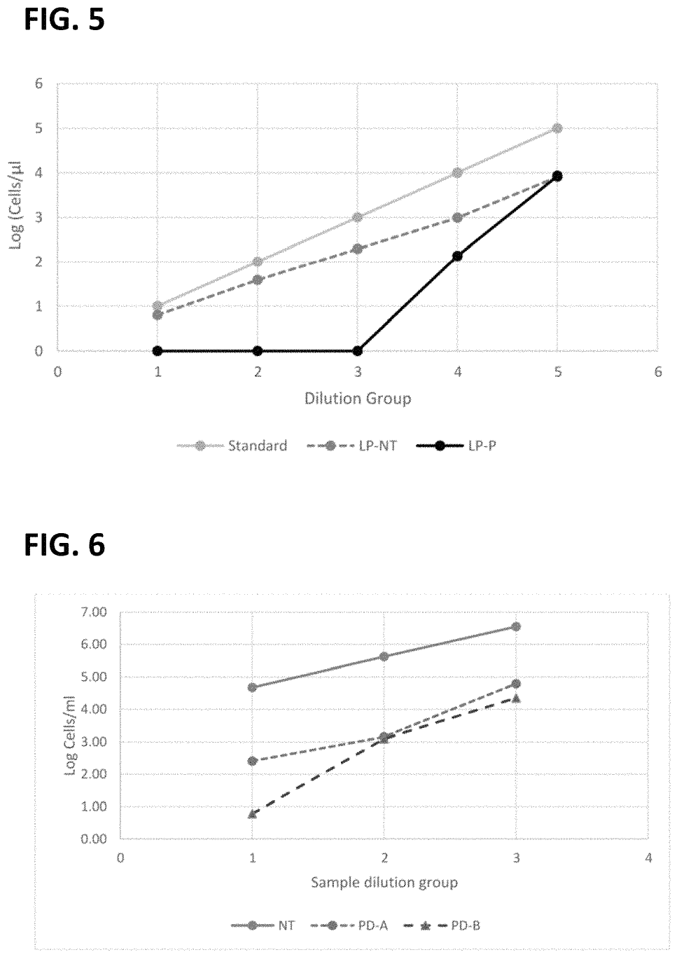

Because sample duplicates were used for PCR setup, average PCR data per test sample was obtained to generate the data in the following Figures. PCR data was used to create FIG. 5 which compares PD treatments among sample groups. FIG. 5 shows that PD treatment resulted in a marked reduction in DNA quantity detected in Legionella sample (LP-P), especially in the lower DNA quantity ranges (10¹ Cells/μl to 10³ Cells/μl). This confirms that 1 μM PD can block PCR amplification of free Legionella DNA molecules. Appropriate PD treatment may be used to the proposed viability testing purposes if the filter is non-DNA binding and non-palladium binding.

### Example 2: Sample Preparation Strategies for Reducing Amplification of DNA from Non-Viable Cells

This example illustrates sample preparation strategies for reducing amplification of DNA from non-viable cells. In these experiments, different protocols were tested for reducing amplification of DNA from non-viable Staphylococcus aureus cells.

In this study, free DNA molecules from confirmed non-viable Staphylococcus were used for testing different sample preparation protocols for reducing PCR amplification of DNA from non-living cells. This study assessed free DNA from dead Staphylococcus cells, and can help identify effective treatments for reducing detection of dead microbial cells.

### Materials and Methods

Test material preparation: The following test samples of previously confirmed dead Staphylococcus aureus cells were prepared: G5=Estimated 10⁶ cells/ml (1:10 dilution of dead Staphylococcus cells at 10⁷ cells/ml); G4=Estimated 10⁵ cells/ml (1:100 dilution of dead Staphylococcus cells); G3=Estimated 10⁴ cells/ml (1:1000 dilution of dead Staphylococcus cells).

Treatment procedures: 380 μl aliquots of each dilution sample were distributed into three tubes for the treatments: NT: samples without PD treatment; no centrifugation. PD-A: samples were treated with 5 μM PD and then shaken at room temperature for 5 minutes without rinsing with H₂O; no centrifugation. PD-B: samples were treated with 5 μM PD and shaken at room temperature for 5 minutes followed by centrifugation at 13,000 rpm for 5 minutes. 370 μl supernatant was discarded and 370 μl H₂O added.

PCR: Each PCR reaction mix contained a set of Staphylococcus aureus-specific primers (forward (SEQ ID NO: 5) and reverse (SEQ ID NO: 6), 1.0 μM each), a Staphylococcus aureus specific probe (0.2 μM, SEQ ID NO: 4), and Life Technology Taqman® Universal Mastermix II (1×). The sequence information for primers and probe can be found in Table 1. The same PCR assay conditions from Example 1 was used.

### Results

Because sample duplicates were used for PCR setup, average PCR data per test sample was obtained to generate the data in the following Figures. PCR data were plotted as a linear graph in FIG. 6. The cell dilution curves across different dilutions show that rinsing steps can reduce the DNA quantity from dead cells with this treatment procedure. PD-A produced the same log difference from untreated (NT) across the three sample groups. This indicates that viability agent PD can reduce the quantity of detectable DNA of dead cells. This shows its utility in viability PCR.

In summary, the data from untreated samples (NT) surprisingly indicates the presence of free amplifiable DNA molecules in dead Staphylococcus cells but shows that PD treatment B can reduce the quantity of this amplifiable free DNA from dead Staphylococcus cells. Some PD treatment thus can increase the efficiency of viability PCR for microorganism testing, but appropriate PD treatment may be used to the proposed viability testing purposes if the filter is non-DNA binding and non-palladium binding. Meanwhile, the direct PCR detection on the free dead cell DNA without extraction provided some insightful information in viability testing and suggested that filtration, alone, could be used for viability PCR.

### Example 3: Effects of H₂O Rinsing of Samples on Dead Microbial Cells (Gram-Positive Microorganism)

This example illustrates the effects of H₂O rinsing of samples on dead microbial cells present in different concentrations, using Staphylococcus cells as a representative Gram-positive microorganism. Goals of PCR-based microbial viability testing include ensuring that viable cells are accurately detected and/or enumerated while reducing or eliminating the quantity of detectable DNA from dead microorganism cells, PCR analysis to detect Staphylococcus bacteria, a Gram-positive bacterium, has generated challenges in other existing viability testing techniques such as PMA PCR, due to the cell membrane structure of Gram-positive bacteria. Data from Example 2 demonstrated that H₂O rinsing can improve viability testing by removing free DNA from samples containing dead microbial cells. In this example, to further investigate the effect of H₂O rinsing, free DNA generated from different rinsing steps was quantified. In these experiments, confirmed non-viable Staphylococcus aureus cells were used to further elucidate the mechanism of, and strategy for, the H₂O rinsing strategy. This study was done with centrifugation to see whether the rinsing steps may help to remove dead cell DNA by centrifugation.

### Materials and Methods

Test material preparation: Obtain previously confirmed dead Staphylococcus aureus cells and prepare for these samples therefrom (in microfuge tubes): C=1 ml of 1:10 dilution of dead Staphylococcus cells (Estimated 10⁸ cells/ml); D=1 ml of 1:100 dilution of dead Staphylococcus cells (Estimated 10⁷ cells/mi); E=1 ml of 1:1000 dilutions of dead Staphylococcus cells (Estimated 10⁶ cells/ml).

Treatment procedure: Four test rinsing procedures were tested: Rinse 0: Centrifuge samples at 4000 rpm for 5 minutes; Transfer 100 μl of the supernatant into new tubes for later PCR use; Discard the remaining supernatant. Rinse 1: Add 800 μl of H₂O into the sample microfuge tubes and mix well; Centrifuge samples at 4000 rpm for 5 minutes; Transfer 100 μl of the supernatant into new tubes for later PCR use; Discard the remaining supernatant; Rinse 2: Add 700 μl of H₂O into the sample microfuge tubes and mix well; Centrifuge samples at 4000 rpm for 5 minutes; Transfer 100 μl of the supernatant into new tubes for later PCR use; Discard the remaining supernatant; Rinse 3: Add 600 μl of H₂O into these empty tubes and mix well; Centrifuge samples at 4000 rpm for 5 minutes; Transfer 100 μl of samples into new tubes for later PCR use.

PCR: Each PCR reaction mix contained a set of Staphylococcus aureus-specific primers (forward (SEQ ID NO: 5) and reverse (SEQ ID NO: 6)) (1.0 μM each), a Staphylococcus aureus specific probe (0.2 μM, SEQ ID NO: 4), and Life Technology Taqman® Universal Mastermix II (1×). The sequence information for primers and probe can be found in Table 1. The same PCR assay conditions from Example 1 was used.

### Results

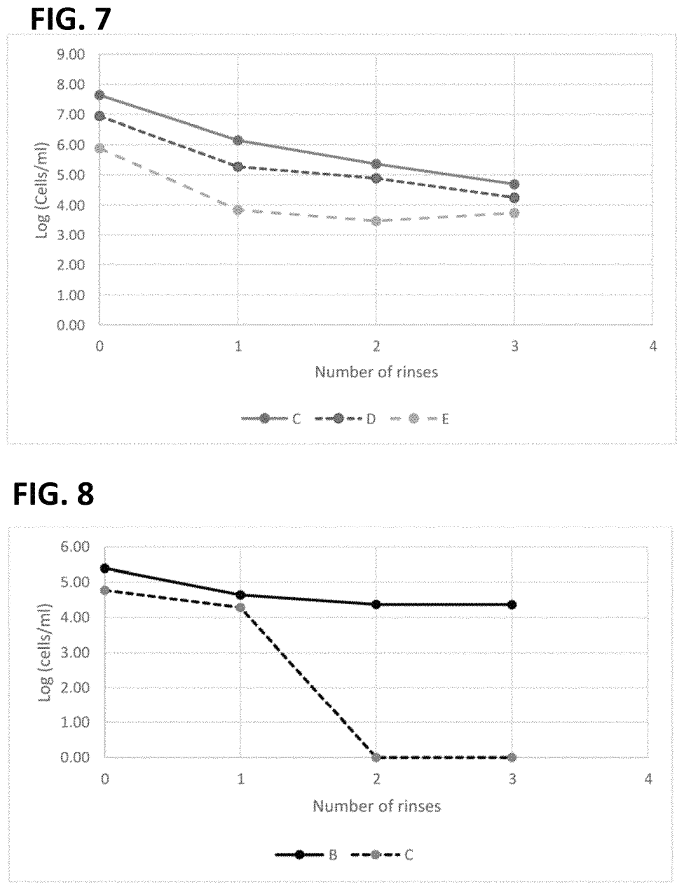

Because sample duplicates were used for PCR setup, average PCR data per test sample was obtained to generate the data in the following Figures. All PCR data was used to generate FIG. 7 to show effects of different rinsing steps. FIG. 7 shows that the samples before rinsing have the highest amount of detectable dead Staphylococcus cell DNA. After the first rinse, average log value reduction was about 1.74. After the second rinse, average log value reduction was 2.25 from the base level. After the third rinse, average log value reduction was 2.60 from the base level. This confirms that H₂O rinsing has a significant effect for a PCR test for microbial viability. The rinsing step may also be useful for the proposed viability testing strategy by removing unwanted free dead cell DNA of Gram-positive bacteria.

### Example 4: Effects of Rinsing Steps on Dead Gram-Negative Bacteria

In this example, confirmed non-viable, Gram-negative Pseudomonas cells were used to test the effectiveness of rinsing strategies on Gram-negative bacteria. This study was done with centrifugation to see whether the rinsing steps could help to remove dead cell DNA by centrifugation.

### Materials and Methods

Test material preparation: Obtain previously confirmed dead Pseudomonas aeruginosa cells and prepare test materials therefrom (in microfuge tubes): B=1 ml of 1:1000 dilution of dead Pseudomonas cells (Estimated 10⁶ cells/ml); C=1 ml of 1:10000 dilution of dead Pseudomonas cells (Estimated 10⁵ cells/ml).

Treatment procedure: Four rinsing procedures were tested. Rinse 0: Centrifuge samples at 4000 rpm for 5 minutes; Transfer 100 μl of the supernatant into new tubes for later PCR use; Discard the remaining supernatant. Rinse 1: Add 800 μl of H₂O into the sample microfuge tubes and mix well; Centrifuge samples at 4000 rpm for 5 minutes; Transfer 100 μl of the supernatant into new tubes for later PCR use; Discard the remaining supernatant; Rinse 2: Add 700 μl of H₂O into the sample microfuge tubes and mix well. Centrifuge samples at 4000 rpm for 5 minutes; Transfer 100 μl of the supernatant into new tubes for later PCR use; Discard the remaining supernatant; Rinse 3: Add 600 μl of H₂O into these empty tubes and mix well; Centrifuge samples at 4000 rpm for 5 minutes; Transfer 100 μl of samples into new tubes for later PCR use.

PCR: Each PCR reaction mix contained a set of Pseudomonas aeruginosa-specific primers (forward (SEQ ID NO: 11) and reverse (SEQ ID NO: 12)) (1.0 μM each), a Pseudomonas aeruginosa specific probe (0.2 μM, SEQ ID NO: 10), and Life Technology Taqman® Universal Mastermix II (1×). The sequence information for primers and probe can be found in Table 1. The same PCR assay conditions from Example 1 was used.

### Results

Because sample duplicates were used for PCR setup, average PCR data per test sample was obtained to generate the data in the following Figures. FIG. 8 confirms that H₂O rinsing is effective for PCR-based quantitation of Gram-negative bacteria (Pseudomonas used as an example). When a lower initial dead cell count is used for testing, a higher number of rinsing steps can reduce the DNA quantity to undetectable levels in PCR alone, as seen in FIG. 8 for the second rinse and the third rinse. These results suggest that appropriate rinsing steps may be useful for viability PCR strategies described herein.

### Example 5: Effects of Rinsing on Non-Viable and Viable Salmonella with a Filter Centrifugal Device

Objectives: to study the effect of sample rinsing on PCR detection of microorganisms by using a filter tube to test both non-viable and viable microorganisms, using Salmonella enterica as a representative microorganism.

Previous examples provided herein show for the first time that several rinsing steps can reduce the concentration or number of PCR amplifiable free DNAs in a sample containing dead bacterial cells. However, rinsing with centrifugation could potentially remove live cells during the rinsing process such as removing supernatant from the sample tubes. In order to prevent this, it was considered that a filter might be able to separate the live cells from free, dead cell DNAs and to retain the live cells after rinsing. Appropriate devices that can serve this function were tested. To determine whether smaller filters could support this type of viability testing and analysis, a filter tube manufactured by Pall Corporation was chosen to as an appropriate example for testing the functionality of the method of the present disclosure.

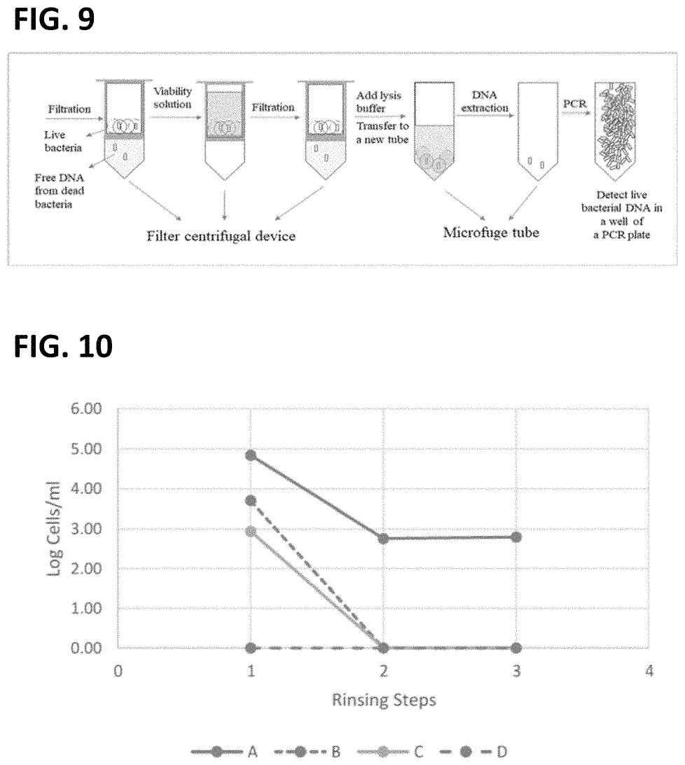

This filter tube is similar to filtration systems for microbiological testing, but has never previously contemplated for PCR analysis or viable PCR testing for microbes. The tube selected for initial testing was the Pall Corporation 0.2 micro nanosep centrifuge tube (ODM02C33) which is composed of a filter and a microfuge tube. Although this tube is not designed for viability PCR testing, this tube can be used as an example of this type of filter arrangement for our study. Importantly, this is the first report on using this type of device for PCR-based viability testing (See FIG. 9).

### Materials and Methods

In this experiment, a filter tube was used to test the rinsing effect on live and dead bacteria. The filtrate at different points in the process was collected to study the DNA quantity at different rinsing steps. The DNA extraction was carried out by using the Mericon DNA Bacteria Kit. PCR data was calculated as cells/ml and the log value of cells/ml was used for data analysis.

Test material preparation: Dead (non-viable) Salmonella sample preparation: Salmonella enterica cultures were heat-killed and confirmed to have no viable CFUs. The dead Salmonella cells were normalized and used at 10⁶ cells/ml. Make four ten-fold dilutions to obtain test samples: Sample A=10⁵ cells/ml; Sample B=10⁴ cells/ml; Sample C=10³ cells/ml; sample D=10² cells/ml. Viable Salmonella materials preparations: Sample E: 1 ml of one-hour fresh Salmonella culture inoculated with two colonies from a culture plate containing pure Salmonella colonies; Sample F: Transfer 100 μl of the one-hour-culture to 900 μl of old culture resuspension (at 4 C for 48 hours) containing unknown number of live and dead cells. Sample G: Transfer 100 μl of sample F to 900 μl of sterile H₂O. Sample H: Transfer 100 μl of sample G to 900 μl of sterile H₂O. Therefore, sample F, sample G and sample H are mixed cell samples.

As this is the first time to test a one-hour culture, the cell count in the bacterial sample was not known. However, this study did not require an accurate, quantified CFU since the goal was to test live cells for the study. It was suspected that the old culture resuspension at 4° C. might have dead cells. Therefore, sample F, G and H might have had an unknown number of dead cells. This same set contains an accurate dilution series of an unknown CFU and mixed growth sample. This imitates a potential real-world scenario example of commercial application for the microbial viability PCR.

Sequential rinsing pretreatment prior to PCR: Rinse 0: Transfer 500 μl of each sample (A-H) into a filter inside a tube. Centrifuge the samples at 13,000 rpm for 5 minutes. Collect 100 μl filtrate from each sample for PCR analysis. Transfer the filters into new tubes. Rinse 1: Add 500 μl H₂O into the filter and mix well. Incubate at 37° C. for 2 minutes in a slow shaker. Centrifuge samples at 13.000 rpm for 5 minutes. Collect 100 μl filtrate from each sample for PCR analysis. Transfer the filters into new tubes. Rinse 2: Add 500 μl H₂O into the filter and mix well. Incubate at 37 C for 2 minutes in a slow shaker. Centrifuge samples at 13,000 rpm for 5 minutes. Collect 100 μl filtrate from each sample for PCR analysis. Transfer the filters into new tubes.

DNA extraction with filters to create test DNA samples: Add 200 μl of FAST LYSIS buffer (available from Qiagen) to each sample filter in a separate tube and mix well with a pipette for each sample suspension. Transfer lysis suspension from filter into the bottom tube and vortex samples for 3 min. Place samples in thermal block at 99° C. for 10 minutes. Cool samples on ice for 2 minutes. Centrifuge at 13,000 rpm for 5 minutes and transfer 100 μl of the top layer to new tubes.

PCR: Each PCR reaction mix contained a set of Salmonella enterica-specific primers (forward (SEQ ID NO: 8) and reverse (SEQ ID NO: 9)) (1.0 μM each), a Salmonella enterica specific probe (0.2 μM, SEQ ID NO: 7), and Life Technology Taqman® Universal Mastermix II (1×). The sequence information for primers and probe can be found in Table 1. The same PCR assay conditions from Example 1 was used.

### Results

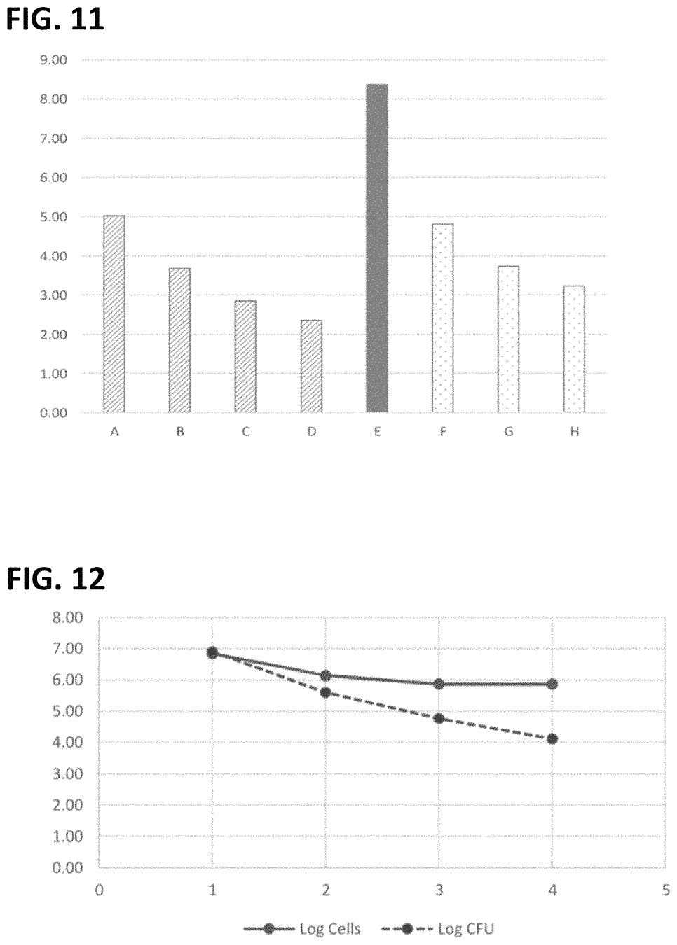

Because sample duplicates were used for PCR setup, average PCR data per test sample was obtained to generate the data in the following Figures. As free DNA can be found from the filtrate samples, data from filtrate samples are presented in FIG. 10 demonstrate that the quantity of free DNA (log cells/ml) from dead cells was dramatically reduced after the first rinse. This is more apparent in samples containing lower initial DNA count (B, C, and D). Data presented in FIG. 11 were obtained from extracted DNA obtained from dead cells (A, B, C, and D), live cells (E), and mixed cells (F, G, H). The figure shows that some dead bacterial cells still have some DNA inside cells after the rinsing treatment. However, the amount was reduced when the dilution of live cell samples increases (see mixed cells: F, G, and H). This further demonstrate the filtration effect in viability testing.

In summary, these experiments show that presence of free dead cell DNA in a sample can be controlled by H₂O rinsing with a filter centrifugal device; High DNA quantity was observed in filtrate separated by the filter before rinsing. This indicates that a significant amount of free DNA from dead cells were passed through the filter and stay in filtrate. This confirms that the filtration step is an important step in viability PCR testing.

### Example 6: DNA Extraction Protocol for Gram Negative Bacteria

Objectives: to validate a lab-developed DNA extraction protocol with a dilution curve for Gram-negative microorganisms, using Salmonella enterica as a representative example organism.

Introduction: In this example, a DNA extraction protocol is described that facilitates PCR-based viability testing. In this study, a filter tube was used as the first step to remove free dead cell DNA before DNA extraction in order to retain a high degree of live cells for the DNA extraction process. Both live cells and mixed cells were tested to compare PCR data for the evaluation of the DNA extraction protocol. Also, this example compared the accuracy of PCR data obtained from these extracted samples with the industry standard for live cell detection (cell culturing).

### Materials and Methods

A filter tube is used in this study. A live culture detection method is used to validate live cell count.

Live culture detection method: The traditional culture method was used to measure the bacterial cell count for each test sample so that the culture data can be compared with the PCR data. When the common pathogens were used for our studies, the LB media was prepared from LB Broth purchased from Neogen Corporation to culture the studied pathogens for cell count verification of the relevant test samples such as live samples or mixed cell samples.

Test Material Preparation: Dead Gram-negative Salmonella enterica cells: concentration 10⁶ cells/ml from heat-killed, normalized stock. Live Salmonella enterica cell stock: As shown in Example 5, free DNA from dead cells was found before rinsing treatment. To concentrate the live cells and remove the dead cells from the suspension, 400 μl of 3-hour culture was subjected to centrifugation at 13,000 rpm for 5 minutes. The supernatant was immediately removed and 400 μl of sterile H₂O was added to resuspend the live cell pellet. The resultant live cell stock contained an unknown number of live cells. The cell count number was subsequently determined by a traditional culture method.

Live Cells for Testing. Live cell materials for testing: Make a series of ten-fold dilutions of live Salmonella cells: 1/100 dilution of the live cell stock, 1/1,000 dilution of the live cell stock, 1/10,000 dilution of the live cell stock, and 1/100,000 dilution of the live cell stock, comprising a total of four live microbial cell test samples generated. 400 μl of each live sample is used for DNA extraction and 100 μl of each live sample is saved for the culture test. Appropriate dilutions are made to each live sample before plating 100 μl of the sample onto a culture plate containing LB media which is composed of LB Broth purchased from Neogen Corporation.

Mixed cell materials for testing: 100 μl of dead Salmonella cells at 10⁶ cells/ml is added to 400 μl of each of the above diluted live cell samples. There is a total of four mixed live and dead cell test samples generated. 400 μl of each mixed cell sample is used for DNA extraction and 100 μl of each mixed cell sample is saved for culture test. Appropriate dilutions are made to each live sample before plating 100 μl of the sample to a culture plate containing LB media. A mixed cell culture can test different ratios of live and dead cells of a test sample, as well as represent a commercial application for detection and enumeration of microorganisms in an unknown sample.

DNA extraction to produce Test Samples (See FIG. 14): Transfer 400 μl of sample suspension into the filter inside a microfuge tube. Centrifuge samples at 13,000 rpm for 5 minutes. Discard filtrate. Add 200 μl of Tris-EDTA buffer (comprising 10 mM Tris-HCl and 1 mM EDTA) into each filter and pipette up and down to mix. Transfer suspension in the filter to a new microfuge tube. Add 200 μl of Tris-EDTA buffer into the same filter and pipette up and down to mix. Transfer suspension from the filter to the first microfuge tube used at beginning of procedure. Discard the filter. Mix the sample tubes in a shaker (VX-2500) at setting of 8 for 2 minutes. Heat samples at 99 C for 10 minutes and cool on ice for 2 minutes. Centrifuge at 13,000 for 5 minutes. Transfer 100 μl of the top layer of the supernatant into new tubes, which comprised the test materials for PCR analysis.

PCR: Each PCR reaction mix contained a set of Salmonella enterica-specific primers (forward (SEQ ID NO: 8) and reverse (SEQ ID NO: 9)) (1.0 μM each), a Salmonella enterica specific probe (0.2 μM, SEQ ID NO: 7), and Life Technology Taqman® Universal Mastermix II (1×). The sequence information for primers and probe can be found in Table 1. The same PCR assay conditions from Example 1 was used.

### Results

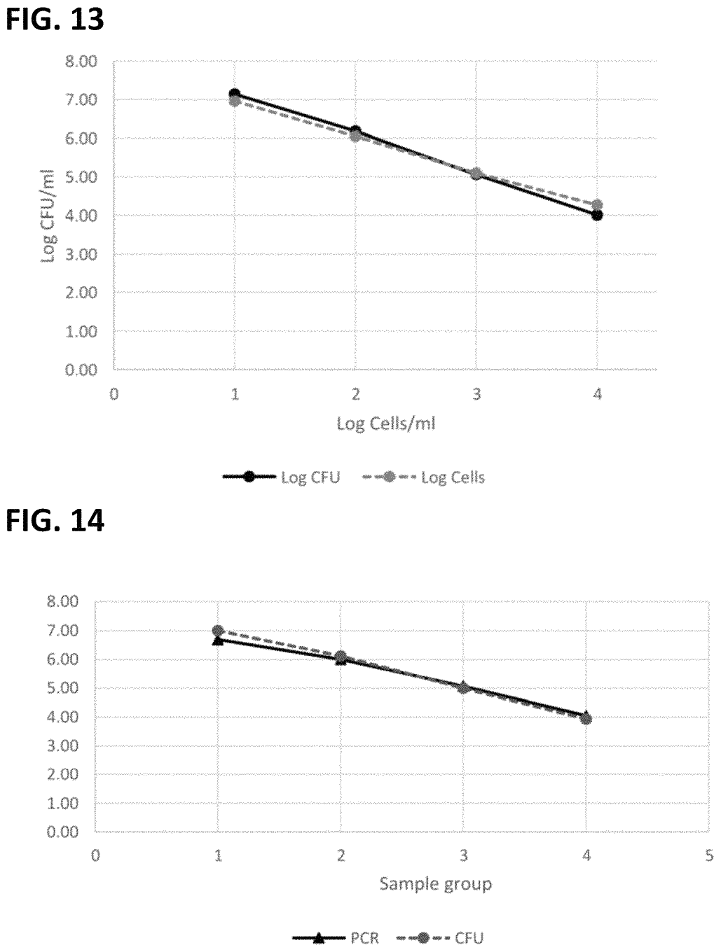

Because sample duplicates were used for PCR setup, average PCR data per test sample was obtained to generate the data in the following Figures. CFU/ml was calculated from the colony number from the culture plate inoculated with each sample according to the dilution used for plating. When the culture data (CFU/ml) and PCR data (cells/ml) are used to create FIG. 12 and FIG. 13, the vertical axis (Y) shows PCR data represented in log cells/ml or culture data in log CFU/ml. The horizontal axis (X) for FIG. 12 and FIG. 13 refers to dilution level: 1=1:100; 2=1:1000; 3=1:10000; 4=1:100000. In FIG. 12, two distinctly different dilution curves are observed between live cells and mixed samples. This is due to the fact that mixed cells contain dead cell DNAs which are at a detectable level by PCR due to no viability treatment. This is very important and until now, limits the utility of, and in many cases prevents, PCR from being used for viable microbe testing. However, the surprising discovery herein shows that using certain rinsing treatments significantly changes this calculus and makes direct PCR assays for viable microbial detection more feasible, and without additional of complex processes and agents used for DNA isolation.

FIG. 13 shows that both culture data (CFU/ml) and PCR data (cells/ml) produce the same dilution curve. This demonstrates the effectiveness of the DNA extraction protocol and validates the DNA extraction protocol described herein. This also provides confidence in future testing and development of microbial PCR viability tests without using the culture method to compare with PCR data. This helps to reduce the testing time and labor costs. This DNA protocol was validated for the viability PCR test for Gram-negative bacteria and could be an alternative DNA protocol for the proposed viability testing strategy.

### Example 7: Effects of Different Rinsing Agents with a Filter Plate

Objectives: To test the use of a filter plate to test different rinsing agents on mixed live and dead cell samples containing different numbers of dead Salmonella enterica cells.

Introduction: In Example 5, potential application of filter tubes was confirmed for viability testing. As filter tubes may not always be feasible for commercial high-throughput PCR testing, such as working with a large sample size, in this example, a more high-throughput option was tested to meet the large-scale sample testing for microbe and pathogen detection. The 96-well filter plate (AcroPrep Advance® 1 ml, P8119, 0.2 μM supor) has been identified as one possible approach for this microbial PCR viability test, even though that product was not designed for this purpose. Other products of similar concepts such as 384-well filter plates and other high-throughput designs should also work. In this example, mixed samples containing two different numbers of dead cells were tested with two different rinsing agents using a 96-well filter plate. This is the first report on the use of 96-well filter plate for viability PCR testing of microorganisms.

### Materials and Methods

Sample preparation: Dead cell materials sources: Salmonella enterica cultures were previously heat-killed and confirmed to have no viable CFUs. The dead Salmonella cells were normalized and used at 10⁶ cells/ml and 10⁴ cells/ml. Live cell sample preparation: Obtain an overnight culture of Salmonella enterica and transfer 500 μl of the culture suspension into a microfuge tube. Centrifuge at 13,000 for 5 minutes. Discard the supernatant and resuspend pellet in 500 μl of H₂O. Prepare the following live samples: A1: 1:100 dilution; A2: 1:1000 dilution; A3: 1:10000 dilution; A4=1:100000 dilution. 400 μl of these diluted live samples were used for viability test and 100 μl of the diluted live samples were saved for culture testing. Appropriate dilutions were made for these samples. before plating 100 μl of the diluted sample onto a culture plate containing LB media. Mixed cell sample preparation: Mixed sample containing 10⁵ cells/ml dead Salmonella cells: Add 100 μl of 10⁶ cells/ml of the dead Salmonella cells into 900 μl of the live cell sample. Mixed sample containing 10³ cells/ml of dead Salmonella cells: Add 100 μl of 10⁴ cells/ml of dead Salmonella cells into 900 μl of the live cell sample. Dead cell sample preparation: 10⁵ cells/ml of dead Salmonella cells: Add 100 μl of 10⁶ cells/ml dead Salmonella cells into 900 μl H₂O. 10³ cells/ml of dead Salmonella cells: Add 100 μl of 10⁴ cells/ml of dead Salmonella cells into 900 μl H₂O.

A 96-well filter plate was used for pretreatment (using protocol below) and then transfer the lysis suspension into microfuge tubes for DNA extraction. Rinsing agents were used for rinsing cells during pretreatment. 5 μM PD and H₂O were used in pretreatment as described in the Pretreatment section below. DNA Extraction is carried out with the DNA extraction protocol as in Example 6 and described below.

Pretreatment: Add 400 μl of sample suspension into the wells of the filter plate. Centrifuge the filter plate at 4000 rpm for 5 minutes. Discard the filtrate and proceed with the following steps. Rinse 1: Add 400 μl of rinsing reagent into the sample wells of the filter plate. Centrifuge at 4000 rpm for 5 minutes. Discard filtrate at the bottom plate. Rinse 2: Add 400 μl of the same rinsing reagent into the sample wells of the filter plate. Centrifuge at 4000 rpm for 5 minutes. Discard filtrate at the bottom plate.

DNA extraction: Add 400 μl of Tris-EDTA buffer (10 mM Tris-HCl and 1 mM EDTA) into each sample well of the filter plate and pipet up and down to mix. Transfer 400 μl suspension from filter plate into individual microfuge tubes. Mix the sample tubes in a shaker (VX-2500) at setting of 8 for 2 minutes. Heat samples at 99° C. for 10 minutes and cool on ice for 2 minutes. Centrifuge at 13,000 for 5 minutes. Transfer 100 μl of top layer into new tubes for PCR.

PCR: Each PCR reaction mix contained a set of Salmonella enterica-specific primers (forward (SEQ ID NO: 8) and reverse (SEQ ID NO: 9)) (1.0 μM each), a Salmonella enterica specific probe (0.2 μM, SEQ ID NO: 7), and Life Technology Taqman® Universal Mastermix II (1×). The sequence information for primers and probe can be found in Table 1. The same PCR assay conditions from Example 1 was used.

### Results

Because sample duplicates were used for PCR setup, average PCR data per test sample was obtained to generate the data in the following Figures. In FIG. 14, the horizontal axis (X) contains the different dilution groups (A1, A2, A3, and A4). Data presented in FIG. 14 demonstrates that the DNA extraction protocol works for detection of live cells from live cell samples as the PCR data closely mirrors the live culture data.

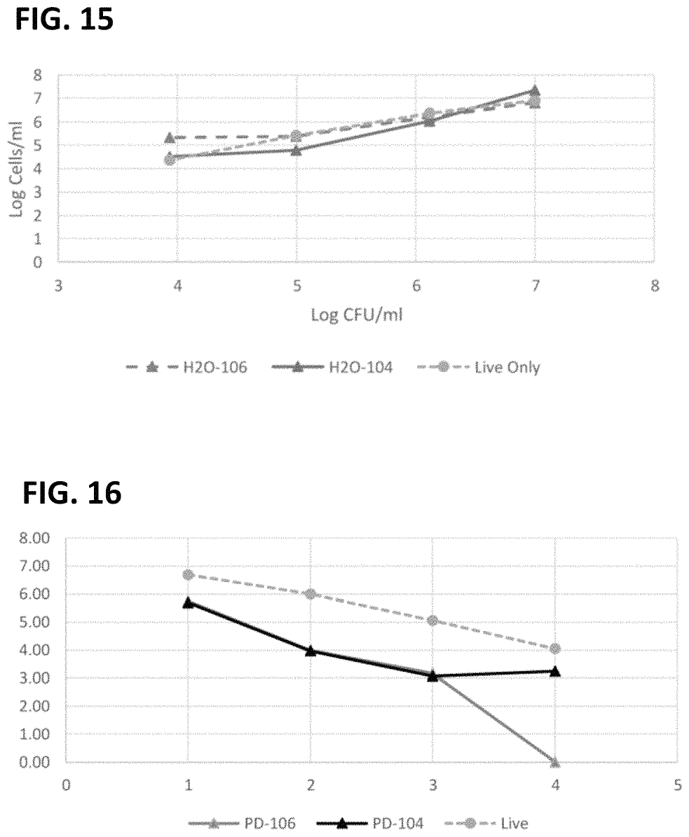

FIG. 15 demonstrates the H₂O rinsing effect on mixed cell samples. When only 10⁴ cells/ml of dead cells are included in the mixed cell samples, the PCR data from the mixed samples show equivalency to the data from live cells by sharing a similar dilution curve. When 10⁶ cells/ml of dead cells are included in the mixed cell samples, the PCR data from mixed samples shows reduced equivalence to the live cell data at lower CFU, such as 10⁴ CFU/ml.

FIG. 16 shows that 5 μM PD rinsing reagent reduced PCR detection of live cells in the mixed cell samples. This may be due to a high PD concentration.

In summary, H₂O rinsing works for detecting live cells when lower number of dead cells are mixed with live cells. 5 μM PD reduce cell count number for both live cells and dead cells. This is the first report on using the 96-well filter plate for high-throughput viability PCR testing. This study also shows a potential negative effect with the use of PD for viability test and suggests that rinsing with H₂O could be an alternative to PD in viability PCR testing.

### Example 8: An Effective PCR-Based Viability Testing Strategy

Objectives: to evaluate the rinsing strategy for microbial PCR viability testing by comparing a filter tube with rinsing and microfuge tube without rinsing on mixed Salmonella enterica samples under different dilutions.

Introduction: In this example, cell numbers in mixtures of live and dead cells were determined. The mixed cell samples were composed of diluted live cell samples and a fixed number of dead cells equivalent to the initial live cell number.

### Materials and Methods

A filter tube is used in this study as an example filtration device. In this experiment, a filter tube type device was chosen to test the rinsing effect on live and mixed bacteria. DNA extraction was done after different pretreatments on different sample groups. Salmonella enterica was used as a model microbe in this study. Different pretreatments are used for different samples. Rinsing agents were PD or H₂O. The specific rinsing treatments used for different test samples are detailed as below. Cicadea Biotech developed the DNA protocol is used for microbial DNA extraction. Data analysis included comparing PCR data (presented in log cells/ml) from mixed cell samples and live cell samples.

Sample Preparation: 1) Cell stock sample preparation: Transfer 500 μl of overnight Salmonella enterica culture into two microfuge tubes. Centrifuge at 13000 rpm for 5 minutes. Discard supernatant. Resuspend the tube with 500 μl of H₂O. 2) Dead cell preparation: Place one of the samples from step 1 at 99 C for 5 minutes. Cool on ice for 5 minutes. This heated samples will be the starting material for the dead Salmonella samples. Make a 1:10 dilution in 5 ml. 3) Live cell preparation: Transfer 500 μl from the remaining step 1 sample into 4.5 ml of H₂O to make 1:10 dilution. Then make a series of three, ten-fold dilutions, in 10 ml. These four samples will constitute the live Salmonella test materials. 4) Mixed cell preparation: Transfer 300 μl of dead cell samples from step 2 into 2,700 μl of live cells. Repeat this for all dilutions of live cells to obtain four mixed cell sample groups for the study.

Test sample types: L-NT (Live samples without viability test treatment): used as a control; M-NT (Mixed sample without viability test treatment: used as a process control; M-P1/P0 (Mixed sample with filter treatment with rinsing): rinsed with 1 μM PD and then H₂O; M-P1/P1 (Mixed sample with filter treatment with rinsing): rinsed with 1 μM PD twice; M-P0/P0 (Mixed sample with filter treatment with rinsing): rinsed with H₂O twice.

Pretreatment of samples without rinsing (L-NT and M-NT): 400 μl of samples (live or mixed) was transferred into microfuge tubes. Centrifuge at 10,000 rpm for 5 minutes. Remove the supernatant.

Pretreatment for samples with rinsing treatment: 400 μl of samples was transferred into filter inside a microfuge tube. Centrifuge at 10,000 rpm for 5 minutes. Discard filtrate and keep filters in their tubes. Rinse 1: Add 400 μl of rinsing reagent onto the filter. Centrifuge at 10,000 rpm for 5 minutes. Discard filtrate and keep filters in their tubes. Rinse 2: Add 400 μl of rinsing reagent onto the filter. Centrifuge at 10,000 rpm for 5 minutes. Discard filtrate and keep filters in their tubes. The rinsing agent was either 1 μM PD or H₂O according to the test sample type.

DNA Extraction: Lysis buffer was added to sample. For samples without rinsing, this involved adding 400 μl Tris-EDTA buffer (10 mM Tris-HCl and 1 mM EDTA) into each microfuge tube containing sample. For samples with rinsing this involved adding 400 μl Tris-EDTA buffer onto filter and mixing, and then transferring each sample to a new microfuge tube. All samples were heated to 99° C. for 10 minutes and then cooled on ice for 2 minutes. Centrifuge at 13,000 rpm for 5 minutes. Then transfer 100 μl of the top layer of the supernatant into new tubes for PCR setup.

PCR: Each PCR reaction mix contained a set of Salmonella enterica-specific primers (forward (SEQ ID NO: 8) and reverse (SEQ ID NO: 9), 1.0 μM each), a Salmonella enterica specific probe (0.2 μM, SEQ ID NO: 7), and Life Technology Taqman® Universal Mastermix II (1×). The sequence information for primers and probe can be found in Table 1. The same PCR assay conditions from Example 1 was used.

### Results

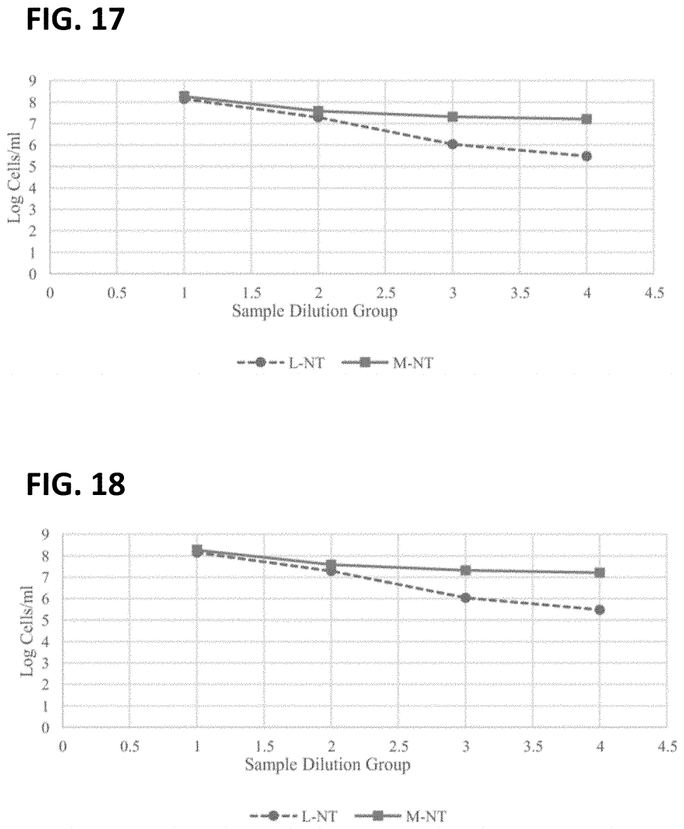

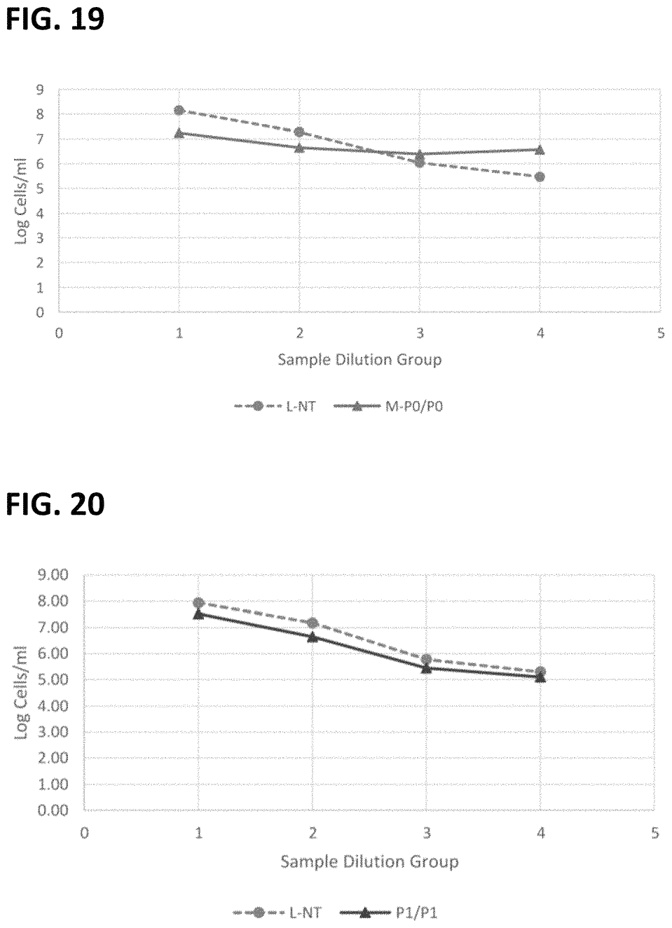

Because sample duplicates were used for PCR setup, average PCR data per test sample was obtained to generate the data in the following Figures. In this example, DNA quantity was measured from PCR data as log Cells/ml. The log values were plotted and compared with different sample groups. Table 2 contains all PCR data from all samples in this example. A pair-wise comparison between different groups of mixed cell samples and live samples without treatment was made to prepare FIG. 17, FIG. 18, FIG. 19 and FIG. 20. The mixed cell samples and live samples without rinsing were compared in FIG. 17 which shows that DNA quantity was not reduced proportionally at lower cell numbers in mixed samples, while DNA quantity from live samples was reduced proportionally at lower cell numbers. FIG. 18, FIG. 19 and FIG. 20 show different dilution curves from the mixed cell samples (rinsed with 1 μM PD/H₂O, H₂O/H₂O, and 1 μM PD/1 μm PD, respectively) and live samples (no rinsing).

TABLE 2
PCR Data (Log Cells/ml) from Different Samples

| Sample | Dilution | L-NT | M-NT | M-PI/P0 | M-PI/PI | M-P0/P0 |
|---|---|---|---|---|---|---|
| 1 | 1:10 | 8.15 | 8.27 | 7.17 | 7.52 | 7.24 |
| 2 | 1:100 | 7.28 | 7.58 | 6.74 | 6.64 | 6.65 |
| 3 | 1:1000 | 6.03 | 7.31 | 6.40 | 5.43 | 6.38 |
| 4 | 1:10000 | 5.47 | 7.20 | 6.16 | 5.09 | 6.57 |

Notes:
Sample information: 1 = 1:10; 2 = 1:100; 3 = 1:1000; 4 = 1:10000.

The DNA quantity from mixed samples was reduced significantly at the high cell count levels and was not reduced proportionally at lower cell count levels. When the mixed samples were rinsed with 1 μM PD twice, the DNA quantity was slightly reduced at the higher cell count level and it was reduced proportionally at lower cell count level. This is shown in FIG. 20 where dilution curve from the mixed samples parallels with the one from live samples. This demonstrates the effectiveness and novelty of a combined PD rinse treatment and filter for microbial PCR viability testing. This is the first report on using filtration process for viability PCR testing. In addition, using 1 μM PD as rinsing agent in a filter system can be used to enhance the viability PCR testing efficiency when higher number of dead cells are present in a test sample. This is the first report on using the combination of PD and filter to achieve effective viability test via PCR for the detection of microbes.

### Example 9: Testing the Filter Effect on Enterococcus and Listeria Genomic DNA

No report has been made for the separation of live bacteria from dead cell DNA using a filtration method. In this experiment, two types of filtration devices were used to test the concept for using filtration as a separation process to enable the detection of live microbial DNA separately from dead microbes or free DNA in a sample. The filtration devices used in this example are a 96-well filter plate (Example: AcroPrep Advance® 1 ml, PN 8119, 0.2 μM supor from Pall Corporation) and filter centrifugal device (ODM02C33 from Pall Corporation). Free DNA from Enterococcus and Listeria that had been previously isolated and quantified on a nanodrop photometer was diluted into test and reference materials with the following concentrations provided in bacterial cell equivalence units: 10⁵ Cells/μl, 10⁴ Cells/μl, 10³ Cells/μl, 10² Cells/μl, 10¹ Cells/μl. Cells/ml is equivalent to genome-copy/ml. This set of diluted test materials was then used in this experiment.

### Materials and Methods:

Test material preparation: 100 μl of each DNA sample (representing: 10⁵ Cells/μl, 10⁴ Cells/μl, 10³ Cells/μl, 10² Cells/μl, and 10¹ Cells/μl) was used for the experiment (Table 9). The dilution series was then used to prepare the following test materials: Standard (STD, only used as a reference standard to quantify other DNA samples); EF-A (Enterococcus faecalis DNA with 96-well filter plate, used as test materials); EF-B (Enterococcus faecalis DNA with filter centrifugal tube, used as test materials); LM-A (Listeria monocytogenes DNA with 96-well filter plate, used as test materials); LM-B (Listeria monocytogenes DNA with filter centrifugal tube, used as test materials).

Treatment procedures: Samples (EF-A and LM-A) were transferred into ten wells of the 96-well filter plate, centrifuged at 4000 rpm for 5 minutes and the filtrate from bottom of the filter plate was used for PCR setup. Samples (EF-B, LM-B) were transferred onto filter of filter tubes, centrifuged at 13000 rpm for 5 minutes and the filtrate from the bottom of the tube was used for PCR setup.

PCR: a set of Enterococcus faecalis-specific primers (forward (SEQ ID NO: 17) and reverse (SEQ ID NO: 18), 1.0 μM each), and a Enterococcus faecalis specific probe (0.2 μM, SEQ ID NO: 16) were used in PCR reactions to detect Enterococcus faecalis; a set of Listeria monocytogenes specific primers (forward (SEQ ID NO: 14) and reverse (SEQ ID NO: 15), 1.0 μM each), and a Listeria monocytogenes specific probe (0.2 μM, SEQ ID NO: 13) were used in PCR reactions to detect Listeria monocytogenes; The sequence information for primers and probe can be found in Table 1. The same PCR assay conditions from Example 1 was used.

### Results:

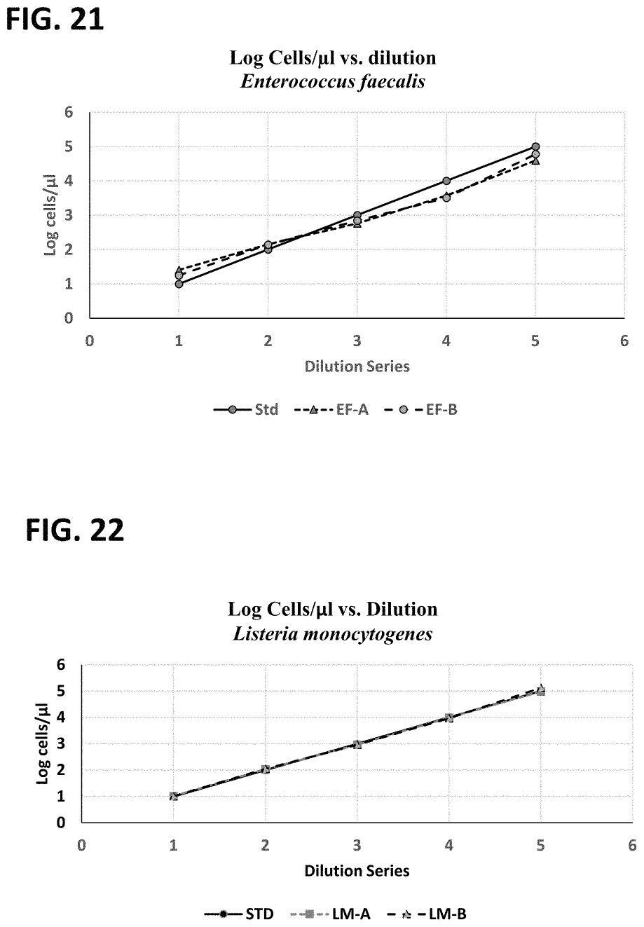

Because sample duplicates were used for PCR setup, average PCR data per test sample was obtained to generate the data in the following Figures. FIG. 21 and FIG. 22, prepared from PCR data of two different PCR assays, respectively, show that the dilution curves from the filtrate products of both the filter plate and the filter tube closely correspond with the dilution curve produced by the standard DNAs. Therefore, both FIG. 21 and FIG. 22 demonstrate the DNA removal efficiency (non-binding pass-through from filter) from either the 96-well filter plate or the filter tubes. This high DNA removal efficiency from the filter is very suitable for our viability testing purpose. This finding provides a basic principle for removing free dead cell DNA from live cells and then later isolating DNA from live cells when lysis buffer is added to break down the live cell membranes. This principle is also based on the use of a non-DNA binding filter with a pore size capable of retaining live bacteria (e.g., 0.2 micron in the test).

### Example 10: Developing an Efficient DNA Extraction Protocol for Testing Viability on Gram Positive Bacteria

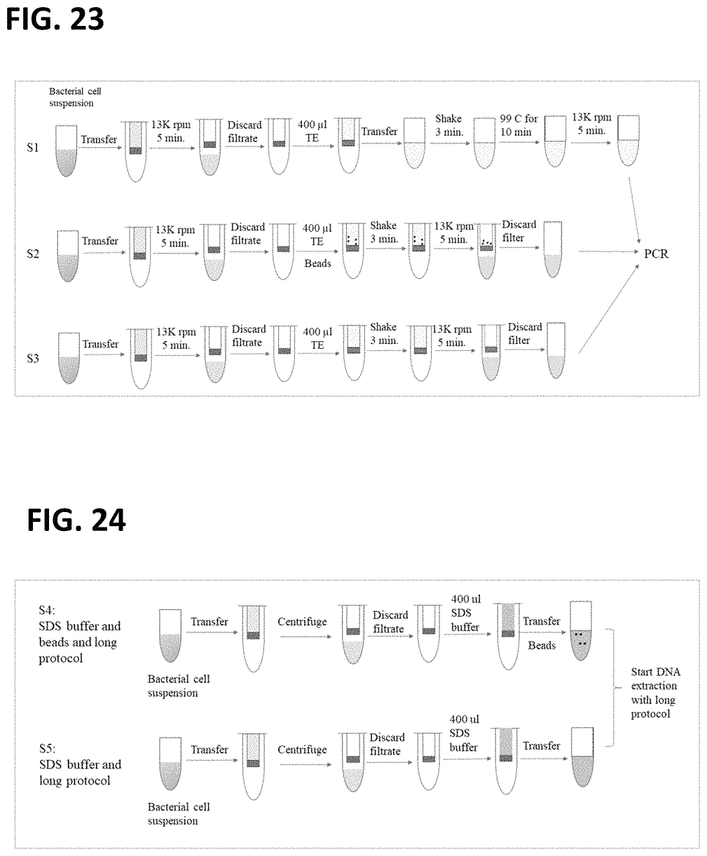

In this example, the effect of glass beads on DNA isolation with both a simplified (short) protocol and long protocol was tested. The test samples were cell suspensions of Staphylococcus aureus. All samples are transferred to filter tubes individually and centrifuged at 13000 rpm for 5 minutes. The filtrates in the bottom tube were discarded. The live cells on the filter and were then processed with different treatments as in the FIG. 23 and FIG. 24.

Throughout Examples 10-18 various lysis buffers and solutions are commonly used. For ease of reference, the composition of these lysis buffers and solutions is described here. SDS buffer is composed of 200 mM Tris-HCl, 20 mM EDTA, 1 M KCl, 1% SDS. TE is Tris-EDTA buffer containing 10 mM Tris-HCl and 1 mM EDTA. KOAc solution (used in various examples to precipitate protein from DNA suspensions) was 5M Potassium Acetate.

### Materials and Methods:

DNA Extraction Protocols. Five different DNA protocols were used to compare their efficiency for viability testing via PCR analysis. Various steps were used as indicated in FIG. 23 and FIG. 24. All of these DNA protocols shared the same filtration step at the beginning to separate and obtain live cells from dead cell DNA via filtration. The filtration was done by centrifugation of the filter tube at 13K rpm for 5 minutes.

DNA Protocol for S1, S2 and S3

S1: After the filtration step, the filtrate was removed. 400 μl TE was added to resuspend live cells on the filter and then the resuspension was transferred to a new microfuge tube. The sample tube was mixed by shaking for 3 minutes and then placed at 99 C for 10 minutes. Followed by centrifugation at 13K rpm for 5 minutes. The top 100 μl is used for PCR.

S2: After the filtration step, the filtrate was removed. 400 μl TE was added to resuspend the live cells on the filter and beads were added into this resuspension. The resuspension in the filter tube was then mixed by shaking for 3 minutes. Followed by centrifugation at 13K rpm for 5 minutes. The filtrate is used for PCR analysis.

S3: After the filtration step, the filtrate was removed. 400 μl TE was added to resuspend the live cells on the filter and the resuspension in the filter tube was mixed by shaking for 3 minutes. Followed by centrifugation at 13K rpm for 5 minutes. The filtrate is used for PCR.

DNA Protocol for S4 and S5

After the filtration step, the filtrate was discarded. 400 μl SDS buffer was added to resuspend the live cells on the filter. Then the resuspension was transferred to a new microfuge tube. In S4 protocol, beads were added into the resuspension while no beads were added into the resuspension for protocol S5. Then the DNA extraction continued with the following protocol:

Samples in microfuge tubes were shaken and mixed at a mixer for 3 minutes. Forty (40) μl of 5M Potassium Acetate was added. The contents were mixed and shaken for 3 minutes and then centrifuged at 13000 rpm for 5 minutes. 300 μl supernatant was transferred into microfuge tubes containing 300 μl isopropanol. Then they were centrifuged at 13000 rpm for 5 minutes. The supernatant was discarded and 300 μl 70% added. The mixture was centrifuged at 13 k rpm for 5 minutes and the supernatant discarded. The pellet was dried at 37° C. for 30 minutes. Then fifty (50) μl TE was added to resuspend the pellet. The suspension was then filtered as shown in FIG. 24 and the resulting filtrate used for PCR.

PCR: Each PCR reaction mix contained a set of Staphylococcus aureus-specific primers (forward (SEQ ID NO: 5) and reverse (SEQ ID NO: 6), 1.0 μM each), a Staphylococcus aureus specific probe (0.2 μM, SEQ ID NO: 4), and Life Technology Taqman® Universal Mastermix II (1×). The sequence information for primers and probe can be found in Table 1. The same PCR assay conditions from Example 1 was used.

### Results:

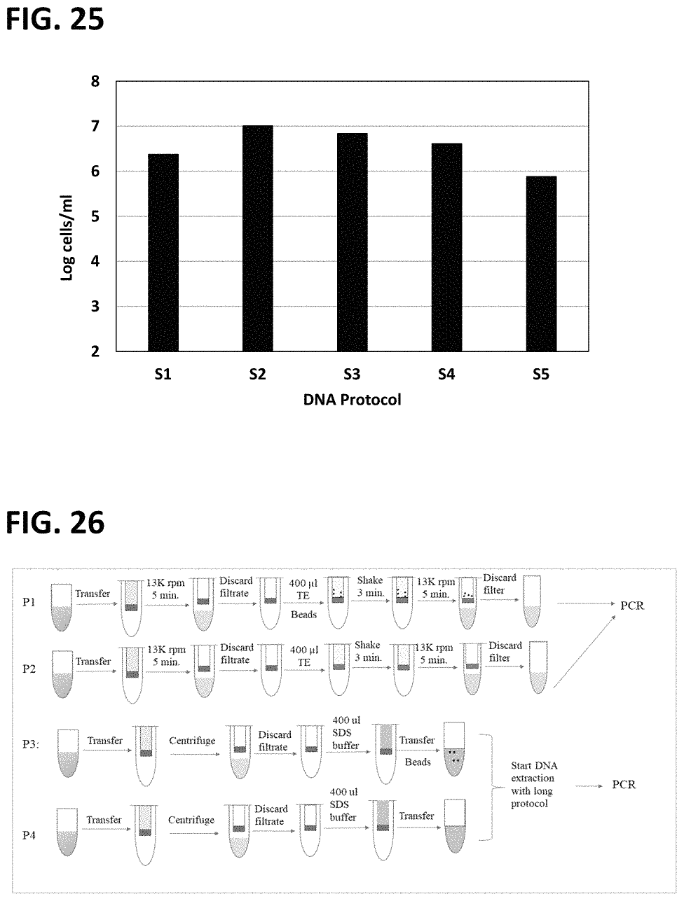

As sample duplicates were used for PCR setup, average PCR data per test sample was obtained to generate the following data in the tables and the graphs. FIG. 25 was generated with PCR data from Table 3.

TABLE 3
PCR Data (Log cells/ml) from Test Samples

| Protocol | | Log |
|---|---|---|
| Name | DNA Protocol Description | cells/ml |
| S1 | TE and beads with heat treatment (Short) | 6.38 |
| S2 | TE and beads and no heat treatment | 7.01 |
| | (with filter) (Short) | |
| S3 | TE and no beads and no heat treatment | 6.84 |
| | (with filter) (Short) | |
| S4 | SDS buffer and beads and long protocol | 6.61 |
| S5 | SDS buffer and long protocol | 5.88 |

FIG. 25 demonstrated that the top three DNA protocols are S2, S3, and S4. S2 and S3 used the same filter to isolate free DNA from disrupted live cells on the filter via centrifugation. S4 used the lysate on the filter and continued with DNA extraction with SDS buffer and other steps as described in the DNA protocol in the “Materials and Methods”. This data showed that S2 produced the highest DNA quantity among all protocols. This confirms the benefits of using beads in isolating DNA from viable Gram-positive bacteria such as Staphylococcus bacteria. In addition, the results also demonstrate that the filter can be used to remove free dead cell DNA from live cells, and to capture the live cells, and to isolate free DNA from live cells after lysis buffer such as TE is applied. This is first report on this simplified DNA protocol to isolate viable microbial DNA from impure samples for PCR analysis.

### Example 11: Developing a DNA Protocol for Legionella Viability Testing

Based on the results from Example 10, a similar experiment was conducted to isolate DNA from Legionella samples (Gram-Negative bacteria). In this example, the effect glass beads had on DNA isolation with both a simplified protocol and a long protocol was tested. Test samples were cell suspensions of Legionella pneumophila. All samples are transferred to filter tubes individually and centrifuged at 13000 rpm for 5 minutes. The filtrates in the bottom tube were discarded. The live cells were assumed to stay on the filter and were processed with different treatments as in the FIG. 26.

### Materials and Methods:

Four DNA protocols are illustrated in FIG. 26. They were tested to compare PCR data and to evaluate the testing efficiency for the detection of DNA from viable Legionella bacteria. These four protocols share the same first step (filtration via centrifugation at 13K rpm for 5 minutes) to capture the live cells on the filter. The filtrates were discarded and they are proceeded with the following processes.

P1 and P2 were the short protocols that used the same filter for DNA isolation from live cells on the filter. After the first filtration step, 400 μl TE was added to the filter to resuspend live cells on the filter and then mixed by shaking for 3 minutes, then a second filtration step was applied to separate DNA from live cells. The filtrate was the final DNA product used for PCR analysis. During this process, beads were added for protocol P1 during TE resuspension of live cells which help to disrupt live cells on the filter and to facilitate the isolation of free DNA from live cells on the filter during the second filtration step. No beads were added for protocol P2. The second filtration step was completed by centrifugation at 13K rpm for 5 minutes.

P3 and P4 were the long protocols that used SDS buffer to lyse live cells on the filter and then the lysed cell resuspensions were into new tubes and went through various steps as follows. Samples in microfuge tubes were shaken and mixed in a mixer for 3 minutes. 40 μl of 5M Potassium Acetate (KOAc solution) was added and the samples mixed and shaken for 3 more minutes. The samples were centrifuged at 13000 rpm for 5 minutes. 300 μl of the supernatant was then transferred into microfuge tubes containing 300 μl isopropanol. The microfuge tubes were centrifuged at 13000 rpm for 5 minutes. The supernatant was discarded and 300 μl 70% ethanol was added to the pellet. The mixture was centrifuged at 13000 rpm for 5 minutes and the supernatant discarded. Pellet was dried at 37° C. for 30 minutes. Fifty (50) μl TE (described below) was added to resuspend the pellet.

PCR: Each PCR reaction mix contained a set of Legionella pneumophila-specific primers (forward (SEQ ID NO: 2) and reverse (SEQ ID NO: 3)) (1.0 μM each), a Legionella pneumophila specific probe (0.2 μM, SEQ ID NO: 1), and Life Technology Taqman® Universal Mastermix II (1×). The sequence information for primers and probe can be found in Table 1. The same PCR assay conditions from Example 1 was used.

### Results:

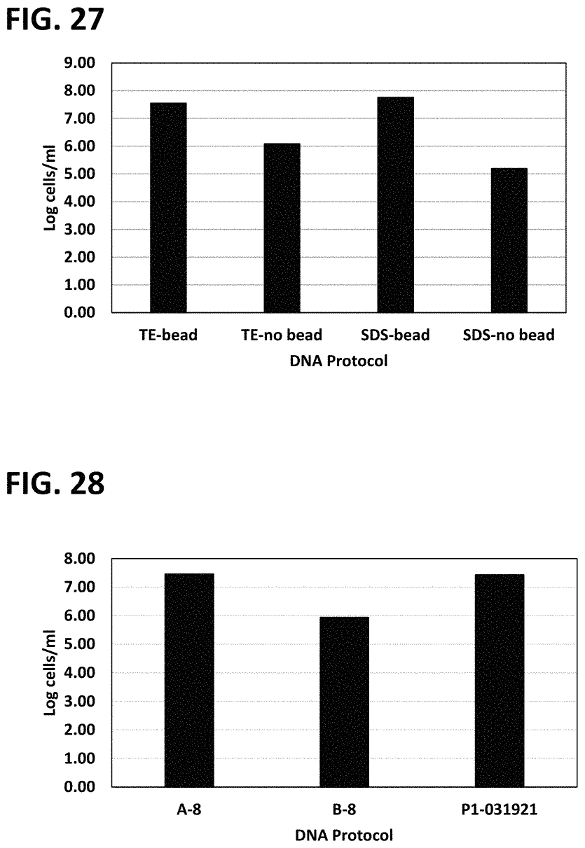

As sample duplicates were used for PCR setup, average PCR data per test sample was obtained to generate the following data in the tables and the figures. FIG. 27 was generated with the PCR data from Table 4.

TABLE 4
Live Cell Count (Log Cells/ml) Comparison among Treatments

| Sample | Protocol | log cells/ml |
|---|---|---|
| P1 | TE-bead | 7.55 |
| P2 | TE-no bead | 6.09 |
| P3 | SDS-bead | 7.76 |
| P4 | SDS-no bead | 5.20 |

FIG. 27 confirmed that protocols with bead treatment produced higher DNA quantity which leads to higher precision in PCR analysis. The PCR data also demonstrated that both the simplified protocol (TE-bead) and the long protocol (SDS-bead) can be used to isolate the live Legionella bacteria after the bead treatment as an equivalent DNA quantity was produced from these two protocols. This is the first report to isolate living Legionella DNA with non-DNA binding 0.2 μm filter.

### Example 12: Effect of Different Volumes of DNA Extraction Buffers on Viable PCR on Microorganisms

To increase the PCR assay sensitivity, a smaller final DNA volume can help to concentrate the final DNA product and increase DNA concentration for PCR analysis. As 400 μl was the final volume of the DNA product from the simplified DNA protocol in Example 10 and Example 11, a smaller final DNA volume was generated to maximize DNA yield. Legionella bacteria was selected for this example.

Materials and Methods:

Sample preparation: 300 μl of Legionella culture suspension was transferred into two filter tubes (A8, B8) and centrifuged at 13000 rpm for 5 minutes. The filtrate was discarded from the tubes and the bottom of the tubes rinsed with 200 μl sterile H₂O (twice). The filter was placed back into the tubes and 200 μl and 100 μl of 1×TE was added to the filter tube A8 and filter tube B8, respectively. Small beads were added to each filter tube and the tubes shaken for 3 minutes. The tubes were centrifuged at 13000 rpm for 5 minutes and the filtrate was collected and used for PCR analysis.

PCR: Each PCR reaction mix contained a set of Legionella pneumophila-specific primers (forward (SEQ ID NO: 2) and reverse (SEQ ID NO: 3)) (1.0 μM each), a Legionella pneumophila specific probe (0.2 μM, SEQ ID NO: 1), and Life Technology Taqman® Universal Mastermix II (1×). The sequence information for primers and probe can be found in Table 1. The same PCR assay conditions from Example 1 was used.

### Results:

As sample duplicates were used for PCR setup, average PCR data per test sample was obtained to generate the following data in the tables and the graphs. FIG. 28 was generated with the PCR data from Table 5. The sample DNA P1 from Example 3 was used at the same PCR experiment to enable to evaluate the efficiency of the DNA protocol with reduced volume.

TABLE 5
Live Cell Count (Loci Cells/ml) Comparison among Treatments

| | | Log |
|---|---|---|
| Protocol | Description | cells/ml |
| A-8 | 200 μl TE | 7.47 |
| B-8 | 100 μl TE | 5.94 |
| P1-031921 | 400 μl TE | 7.44 |

The data from FIG. 28 demonstrated that 200 μl TE buffer produced higher DNA quantity than 100 μl TE. When comparing the 200 μl TE with the sample isolated with 400 μl TE in Example 11, the PCR data was very close and essentially equivalent. Therefore, the short DNA protocol with 200 μl TE is validated for isolating DNA for viability testing experiments.

### Example 13: Viability PCR Test for Gram-Positive Bacteria

In this example, bacterial suspensions from three different Gram-Positive bacteria were obtained separately and used to prepare test samples. A series of 10-fold dilution of live cells was made to produce a set of three 10-fold dilutions on the live cells. These diluted bacterial suspensions were then used to prepare for two types of test samples: live cell only samples (L), mixed samples contain live cells and dead cells or free DNA from dead cells (M). Each mixed sample contained the same amount of dead cell DNA although it may have different dilutions of live cells. The filtration method was used as a viability treatment in this example. Two DNA protocols were used to isolate the live cell DNA from the same test samples and results were compared to determine the efficiency of the viability PCR test.

### Materials and Methods:

Sample sources: The bacterial suspensions used in this example were from these Gram-Positive bacteria: Enterococcus faecalis (EF), Listeria monocytogenes (LM), and Staphylococcus aureus (SA).

Live cell sample preparation. Each bacterial suspension was centrifuged at 4500 rpm for 8 minutes. The supernatant was discarded, and the pellet was resuspended with 1.5 ml of sterile H₂O. Then the resuspension was filtered with filter tube via centrifugation to capture the live cells. The captured live cells were then resuspended with H₂O on the filter and subsequently transferred to a new tube and was then used as the live cell stock. The volume of the live cell stock was adjusted to higher volume to meet the testing needs. Subsequently, two 10-fold dilutions were made from the live cell stock. As a result, three 10-fold dilutions per bacterium was made for the live cell stock in this example.

Preparation of free DNA from dead bacteria: A portion of the live cell stock was transferred to a filter tube and centrifuged at 13K rpm for 5 minutes. The filtrate was removed and 500 μl TE and glass beads were added to the filter inside the filter tube. The sample tube was then shaken and mixed for 3 minutes and followed by a centrifugation at 13K rpm for 5 minutes. The filtrate was transferred to a new tube and used as the dead cell stock. This dead cell stock may be diluted to the right amount of volume to meet the testing needs. Same amount of dead cell stock was applied to different diluted live cell samples to produce mixed cell samples used for the viability test experiment.

Test sample preparation. Test samples for EF bacteria, LM bacteria and SA bacteria were prepared separately. Each mixed cell sample was composed of a live cell sample and a dead cell DNA sample (e.g., 200 μl of live sample of different dilutions and 200 μl of the same dead cell stock were combined in each mixed cell sample). Different diluted live cell samples were combined with a fixed amount of dead cell stock to produce a set of mixed samples for this study. Three types of test samples were prepared in this example: L samples: live cell samples at three 10-fold dilutions and they were labeled as L1, L2, and L3; MF samples: mixed cell samples used for viability testing treatment and labeled as MF1, MF2, and MF3; and M samples: mixed cell samples used as a test control without viability testing treatment and labeled as M1, M2, and M3. Both L and MF samples were placed in filter tubes. M samples were placed in microfuge tubes and served as a process control.

Viability test treatment: Filter tubes were used for viability test in this example. The viability testing process is illustrated as in diagram (FIG. 29). Both L and MF samples were placed in different filter tubes and centrifuged at 13K rpm for 5 minutes. The filtrate containing dead cell DNA in test sample was discarded. The bottom tube was rinsed with sterile H₂O twice. The filter was kept in the same tube and DNA extraction proceeded as follows.

DNA extraction: Two protocols were performed to extract DNA (short and long protocol).

Extraction with filter tube samples (short protocol): 200 μl TE and beads were added to the filter inside the filter tube and shaken for 3 minutes. The sample were then centrifuged at 13K for 5 minutes. The filter was discarded and the filtrate is the DNA product from this short protocol. 20 μl of the filtrate was set aside for PCR analysis and the remaining 180 μl filtrate was used for DNA extraction with the long protocol.

Extraction with microfuge tube samples (long protocol): As the same sample was used for two different DNA protocol, “—B” was added to the sample name such as L or MF to help to identify the DNA samples isolated from the long DNA protocol. For example, L1-B or F-B, etc. 500 μl SDS extraction buffer (defined above) was added to the samples prepared using the short protocol (from extraction “a”) and 300 μl SDS extraction buffer was added to samples in the microfuge tubes (M1, M2, and M3). Small glass beads (2-3 mm) were added to the tubes and shaken for 3 minutes. The tubes were centrifuged at 13000 rpm for 5 minutes before 70 μl of 5M potassium acetate solution was added. The samples were shaken again for 3 minutes and then centrifuged at 13000 rpm for 5 minutes. 650 μl of the supernatant was transferred into new tubes containing 650 μl isopropanol. The new tubes were then centrifuged at 130000 rpm for 3 minutes and the supernatant discarded. 400 μl 70% ethanol was added to the pellet and the mixture centrifuged again at 130000 rpm for 3 minutes. The supernatant was discarded and all liquid emptied completely from the tubes. The pellet was dried at 37° C. for 30 minutes and then 50 μl of 0.1 TE buffer was added to each sample. These final samples were then used for PCR analysis.

PCR: a set of Enterococcus faecalis-specific primers (forward (SEQ ID NO: 17) and reverse (SEQ ID NO: 18), 1.0 μM each), and a Enterococcus faecalis specific probe (0.2 μM, SEQ ID NO: 16) were used in PCR reactions to detect Enterococcus faecalis; a set of Listeria monocytogenes specific primers (forward (SEQ ID NO: 14) and reverse (SEQ ID NO: 15), 1.0 μM each), and a Listeria monocytogenes specific probe (0.2 μM, SEQ ID NO: 13) were used in PCR reactions to detect Listeria monocytogenes; a set of Staphylococcus aureus-specific primers (forward (SEQ ID NO: 5) and reverse (SEQ ID NO: 6), 1.0 μM each), and a Staphylococcus aureus specific probe (0.2 μM, SEQ ID NO: 4) were used in PCR reactions to detect Staphylococcus aureus. The sequence information for primers and probe can be found in Table 1. The same PCR assay conditions from Example 1 was used.

### Results:

As sample duplicates were used for PCR setup, average PCR data per test sample was obtained to generate the following data in the graphs. Dilution curves from different treatments were compared for each bacterium. In addition, the results from the short DNA protocol and long protocol were also obtained to evaluate the efficiency for viable PCR. As the same sample was used for two different DNA protocol, “—B” was added to the sample name such as L or MF to help to identify the DNA samples isolated from the long DNA protocol.

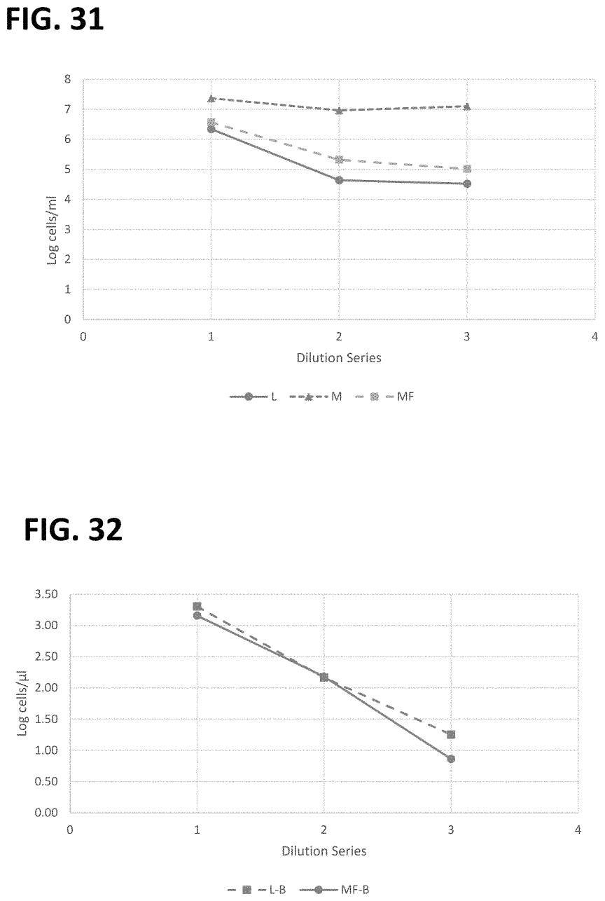

The data from FIG. 30 and FIG. 31 show that the mixed cell samples without filter treatment produce a higher extrapolated cell count per ml when comparing the mixed cell samples with filter treatment. Data presented in FIG. 30 was from the PCR analysis on DNA isolated with the long protocol and data presented in FIG. 31 was from the PCR analysis on DNA isolated with the short protocol. Both protocols show similar trends that the mixed cell samples without filtration produce higher false positives than the ones treated with the filtration. In addition, this is the first report using a filter (non-DNA binding) with a submicron pore size (e.g. 0.2 μm) to isolate DNA from live cells such as Enterococcus faecalis (a Gram-positive bacterium) and separate the live DNA from DNA present from dead cells or free DNA in a sample.

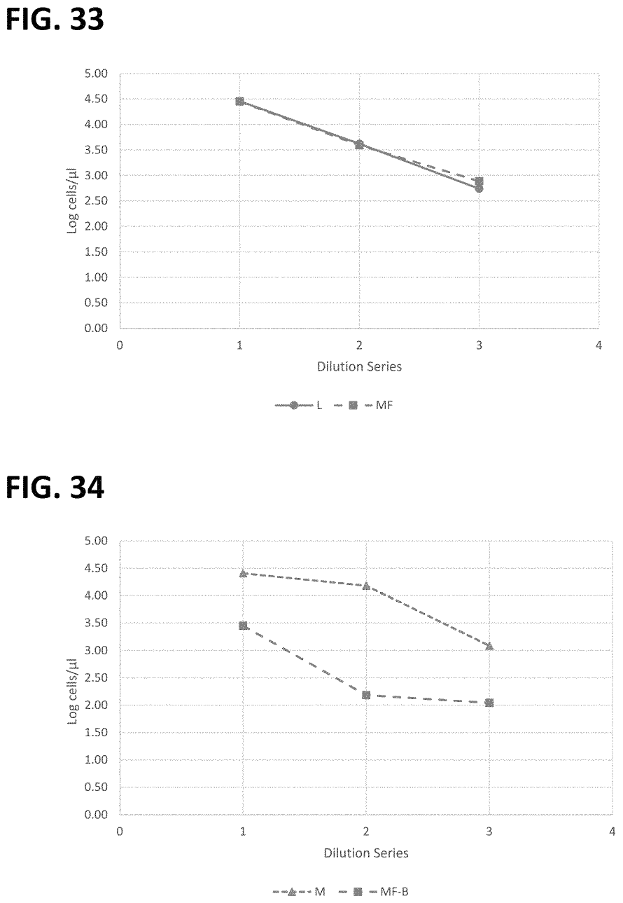

Both FIG. 32 and FIG. 33 show the dilution curves of filtered live cell DNA samples (L) and the filtered mixed samples (MF) match very well with either short protocol (FIG. 33) or long protocol (FIG. 32). This further demonstrated the functionality of the proposed viability test using the short DNA preparation protocol. In addition, this is the first report using a filter (non-DNA binding) with a submicron pore size (e.g., 0.2 μm) to isolate DNA from live cells such as Listeria monocytogenes (a Gram-positive bacterium) separately from DNA present from dead cells or free DNA in a sample.

The data from FIG. 34 demonstrated that mixed samples (M) without filtration can produce higher false positive results when compared with mixed samples (MF-B) with filtration. This is the first report in using a filter to isolate DNA from live Staphylococcus cells.

Overall, this example demonstrated that the proposed viability testing strategy can be applied to detect viable Gram-Positive bacteria such as Enterococcus, Listeria, and Staphylococcus. In addition, the DNA isolation from live cell bacteria with the filter was very innovative and provide a great alternative for bacteria screening process, especially for food testing or water testing areas.

### Example 14: Viability Test on Escherichia Bacteria

In this example, live cell samples from Escherichia coli (EC) were obtained through the filtration method and they were used to prepare for three types of test samples: live cell only samples (EL), mixed samples for viability test (EMF), and mixed samples as process control (EM). Two DNA protocols were used to isolate the live cell DNA and mixed cell DNA after the filtration and the results were compared to determine the efficiency of the viability PCR test.

### Materials and Methods:

Sample sources: The bacterial suspensions used in this example were from Escherichia coli suspension.

Live cell sample preparation: The bacterial suspension was centrifuged at 4500 rpm for 8 minutes. The supernatant was discarded, and the pellet was resuspended with 1.5 ml of sterile H₂O. Then the resuspension was filtered with filter tube via centrifugation to capture the live cells. The captured live cells were then resuspended on the filter and subsequently transferred to a new tube and was then used as the live cell stock. The volume of the live cell stock was adjusted to higher volume to meet the testing needs. Subsequently, two 10-fold dilutions were made from the live cell stock. As a result, three 10-fold dilutions per bacterium was made for the live cell stock in this example.

Preparation of free DNA from dead EC bacteria: 500 μl the live cell stock was transferred into a microfuge tube and centrifuged at 13K rpm for 3 minutes. The supernatant was discarded, 200 μl TE was added to the sample and the sample shaken for 3 minutes before being treated by heat at 99° C. for 10 minutes. The sample was then cooled on ice for 3 minutes and become the dead EC stock for the experiment

Preparation of all test samples: Each mixed cell sample was composed of a live cell sample and a dead cell DNA sample (e.g., 200 μl of live sample of different dilutions and 200 μl of the same dead cell stock were combined in each mixed cell sample). Different diluted live cell samples were combined with a fixed amount of dead cell stock to produce a set of mixed samples for this study. Three types of test samples were prepared in this example: EL samples: live cell samples at three 10-fold dilutions and (labeled as EL1, EL2, and EL3); EMF samples: mixed cell samples used for viability testing treatment (labeled as EMF1, EMF2, and EMF3); and EM samples: mixed cell samples used as a test control without viability testing treatment (labeled as EM1, EM2, and EM3). Both EL and EMF samples were placed in filter tubes. EM samples were placed in microfuge tubes and served as a process control.

Viability test treatment: Filter tubes were used for viability test in this example. The viability testing process was described in Example 13 (e.g., FIG. 29). Both EL and EMF samples were placed in different filter tubes and centrifuged at 13K rpm for 5 minutes. The filtrate containing dead cell DNA in test sample was discarded. The bottom tube was rinsed with sterile H₂O twice. The filter was kept in the same tube and DNA extraction proceeded as follows.

DNA extraction: The same protocols (short and long protocol) described in Example 13 were used for DNA extraction. As the same sample was used for two different DNA protocol, “—B” was added to the sample name such as EL or EMF to help to identify the DNA samples isolated from the long DNA protocol

PCR: Each PCR reaction mix contained a set of Escherichia coli-specific primers (forward (SEQ ID NO: 17) and reverse (SEQ ID NO: 18)) (1.0 μM each), an Escherichia coli specific probe (0.2 μM, SEQ ID NO: 16), and Life Technology Taqman® Universal Mastermix II (1×). The sequence information for primers and probe can be found in Table 1. The same PCR assay conditions from Example 1 was used.

### Results:

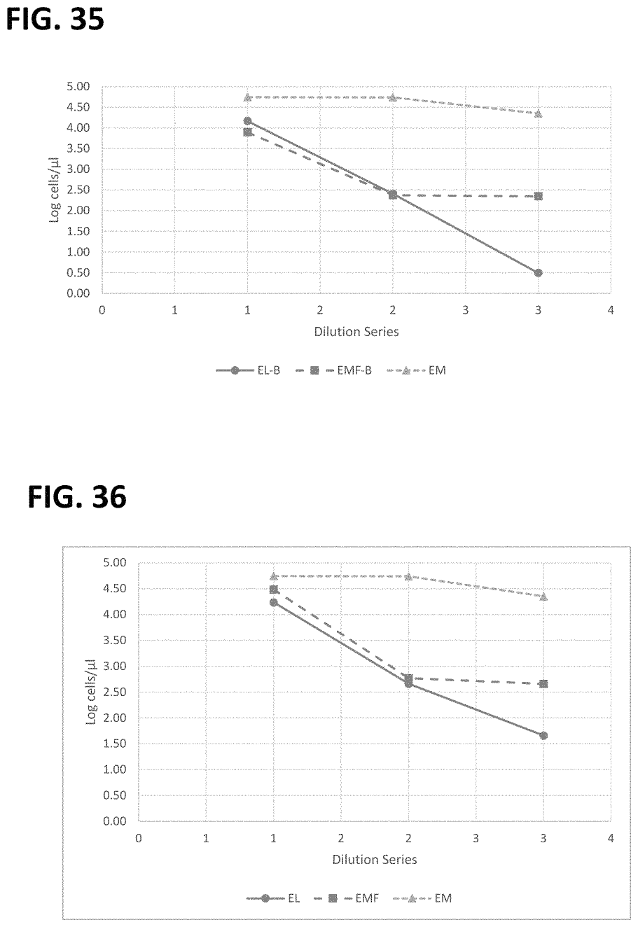

As sample duplicates were used for PCR setup, average PCR data per test sample was obtained to generate the following data in the tables and the graphs. FIG. 35 and FIG. 36 were generated with the PCR data from samples isolated from two different DNA protocols respectively.

The results from FIG. 35 and FIG. 36 demonstrated the effectiveness of the proposed viability testing strategy on E. coli. The dilution curve of the mixed samples (EMF and EMF-B) treated with filtration show a downward trend at higher diluted samples. The dilution curve of the mixed samples (EMF and EMF-B) matched the live samples (EL and EL-B) better than mixed samples without filtration (EM and EM-B). This is the first report using the filter strategy to perform viability test on E. coli samples. This strategy will be very useful for detecting live E. coli from food or water samples that could contain killed, dead, or free DNA which would otherwise confound the results.

### Example 15. Viability Test on Salmonella enterica

In this example, an overnight culture from Salmonella enterica (SE) was used directly to prepare three types of test samples: live cell only samples (L), mixed samples for viability test (F), and mixed samples as process control (M). Two DNA protocols were used to isolate the live cell DNA and mixed cell DNA after the filtration and the results were compared to determine the efficiency of viability test. A 24-well filter plate and vacuum were used in this experiment.

### Materials and Methods:

Sample sources: The bacterial suspensions used in this example were from a Salmonella enterica suspension.

Live cell sample preparation: Six (6) ml of SE overnight culture was transferred to a 50 ml tube containing 24 ml of H₂O and labeled as X1. The tubes were mixed well and then 5 ml of X1 was transferred into a 50 ml tube containing 15 ml of H₂O (labeled as X2). Then 2 ml of X2 was transferred into 18 ml H₂O to make a 1/10 dilution of X2 and labeled as X3. 2 ml of X3 was transferred into 18 ml H₂O to make a 1/100 of X2 and labeled as X4.

Dead SE sample preparation: A previously quantified SE dead cell suspension (10⁵ cells/μl) was used.

Test sample preparation: Each mixed cell sample was composed of a live cell sample and a dead cell DNA sample (e.g., 4000 μl of live sample of different dilutions and 500 μl of the same dead cell stock were combined in each mixed cell sample). Different diluted live cell samples were combined with a fixed amount of dead cell stock to produce a set of mixed samples for this study. Three types of test samples were prepared in this example: L samples: live cell samples at three 10-fold dilutions (labeled as L1, L2, and L3); F samples: mixed cell samples used for viability testing treatment (labeled as F1, F2, and F3); and M samples: mixed cell samples used as a test control without viability testing treatment (labeled as M1, M2, and M3). Both L and F samples were placed in different wells of the 24-well filter plate. M samples were placed in 15 ml conical tube and served as a process control.

Preparation of M samples: M samples were centrifuged in 15 ml tube at 5000 rpm for 10 minutes, the supernatant was discarded, and 400 μl TE was added to resuspend the pellet. The resuspension was then transferred into microfuge tubes.

Viability test treatment with filter plate on L and F samples. L and F samples were prepared in a 24 well filter plate. The 24-well filter plate was then placed on a second 24 well plate on a manifold connected with a vacuum pump. The vacuum was turned on to remove liquid inside the filter plate. The filtrate in the bottom plate was discarded and 1 ml H₂O added into the sample wells of the bottom plate. The bottom plate was spun for a short time and then the H₂O discarded. Another 1 ml of H₂O was added to the sample wells of the bottom plate before they were spun down briefly and the H₂O discarded. The filter plate was reassembled with the bottom plate and 400 μl TE was added to the sample wells (A1 to D1) of the filter plate. Small glass beads (2-3 mm) were added to the wells of the filter plate and the entire plate covered with aluminum foil. The filter plate complex was transferred to a mixer and mix/shaken for 3 minutes. The filter complex was placed back on the manifold connected with a vacuum which was used to transfer the contents of the filter plate into the bottom plate. The filtrate from the bottom plate was transferred to microfuge tubes. 20 μl of the suspension from each sample were then transferred into a 96 well plate for PCR. The remaining suspension was saved in the microfuge tubes for the long DNA protocol described below.

DNA extraction with microfuge tube samples (“-B” was added to the sample name such as L or F to help to identify the DNA samples isolated from the long DNA protocol). 300 μl of SDS extraction buffer was added to all samples. Small beads were added to M samples, but not L and F samples. DNA extraction proceeded as described in Example 13 (long protocol) to isolate DNA from L-B, F-B, and M samples.

PCR: Each PCR reaction mix contained a set of Salmonella enterica-specific primers (forward (SEQ ID NO: 8) and reverse (SEQ ID NO: 9), 1.0 μM each), a Salmonella enterica specific probe (0.2 μM, SEQ ID NO: 7), and Life Technology Taqman® Universal Mastermix II (1×). The sequence information for primers and probe can be found in Table 1. The same PCR assay conditions from Example 1 was used.

### Results:

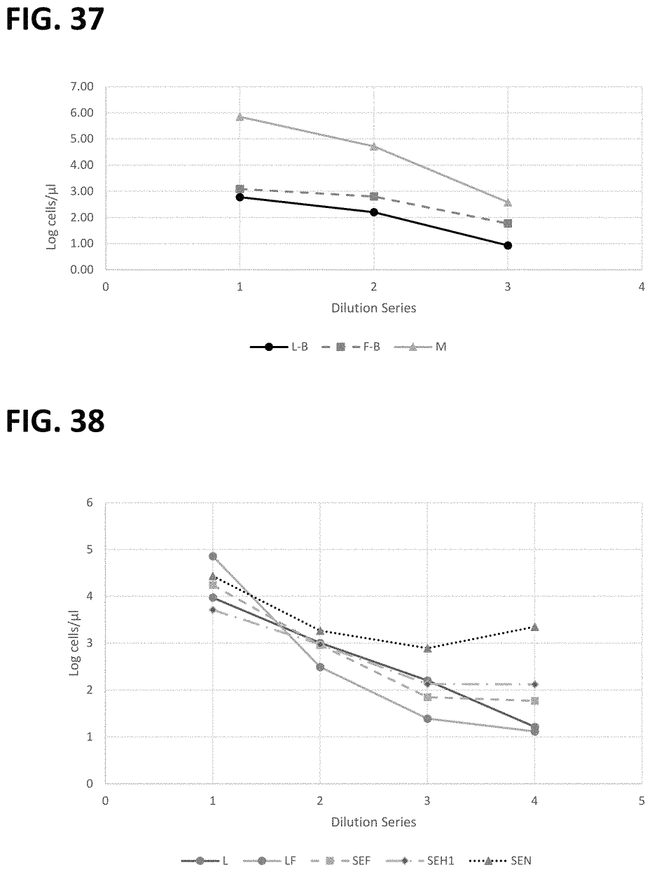

As sample duplicates were used for PCR setup, average PCR data per test sample was obtained to generate the following data in the tables and the graphs. FIG. 37 was generated with the PCR data from Table 6. Due to low SE cell density was in the test samples, the short protocol did not generate sufficient PCR data for analysis. In FIG. 37, L-B and F-B refer to DNA extracted from L and F samples with Long protocol. The data obtained from the long protocol show the significant differences between mixed samples without filtration and samples (mixed or live) with filtration. This is the first report using the filtration method to isolate viable Salmonella bacteria, a Gram-negative bacterium.

TABLE 6
Log cells/μl comparison

| | L-B | F-B | M |
|---|---|---|---|
| 1 | 2.78 | 3.09 | 5.85 |
| 2 | 2.20 | 2.80 | 4.71 |
| 3 | 0.93 | 1.77 | 2.58 |

### Example 16: Viability Test on Salmonella Bacteria

In this example, live Salmonella cells were retained on the 24-well filter plate, then lysis buffer was added to resuspend the live cells and the sample suspension was then transferred into a new microfuge tube where heat treatment was used to isolate DNA from Salmonella enterica (SE), a Gram-negative bacterium. Four 10-fold dilutions of test samples were created to compare the dilution curves for live samples, mixed samples with filter plate and mixed samples without filter plate. In addition, the rinsing treatment was used to compare its effectiveness in removing free DNA from the filter. A vacuum was used for viability treatment with the filter plate.

### Materials and Methods:

Sample sources: The bacterial suspensions used in this example were from a Salmonella enterica suspension.

Live sample preparation: Four (4) microfuge tubes containing 800 μl of the 4-hour SE culture were combined into a 15 ml tube and mixed well. Sterile H₂O was added to bring the suspension up to 10 ml and labeled as X1. Then three 10-fold dilutions were made to produce X2 (1/10 of X1), X3 (1/100 of X1), and X4 (1/1000 of X1). The final volume for X1 was 40 ml and X2, X3, and X4 were each 30 ml.

Dead cell preparation: 7 ml of X1 was transferred into a 15 ml tube and heated at 99° C. for 10 minutes; cooled on ice for 3 minutes; and used as dead stock for mixed samples.

Test sample preparation: Each mixed cell sample was composed of a live cell sample and a dead cell DNA sample (e.g., 4000 μl of live sample of different dilutions and 400 μl of the same dead cell stock were combined in each mixed cell sample). Different diluted live cell samples were combined with a fixed amount of dead cell stock to produce a set of mixed samples for this study. Five types of test samples were prepared in this example: (1) L samples containing only live cell samples with different dilutions without filtration treatment (labeled as L1, L2, L3, and L4). They were placed in 15 ml tube. (2) SEN samples were mixed cell samples without filtration treatment and labeled as SEN1, SEN2, SEN3, and SEN4. They were in 15 ml tube. They were used as a process control. (3) SEF samples: Mixed cell samples used for viability treatment and labeled as SEF1, SEF2, SEF3, and SEF4. They were placed in a 24-well filter plate. (4) SEH-1 samples: Mixed cell samples used for viability treatment with rinsing treatment with H₂O and labeled as SEH1, SEH2, SEH3, and SEH4. They were placed in a 24-well filter plate. (5) LF samples: Live cell only samples used for viability treatment and labeled as LF1, LF2, LF3, and LF4. They were placed in a 24-well filter plate.

Preparation of microfuge tube samples: Centrifuge samples in 15 ml at 5000 rpm for 10 minutes; discard supernatant and add 400 μl TE to resuspend the pellet; save them for DNA extraction later.

Viability test with filter plate treatment. Samples were prepared as described herein and placed inside wells of a 24 well filter plate on a 24 well plate on a device connected with a vacuum pump (as described in Example 14). The vacuum was turned on to remove liquid inside the filter plate and the filtrate was discarded from the bottom plate. 400 μl of TE was added to sample (A1 to D1; A3 to D3) and the suspension transferred into new microfuge tubes. For SEH samples with a rinsing treatment, 1 ml H₂O was added to each well (A2 to D2) and the vacuum turned on again to remove the liquid. The filtrate was discarded. 400 μl of TE was added to sample (A2 to D2) and the suspension transferred into new microfuge tubes.

DNA extraction. The samples in the microfuge tubes were mixed in the shaker for 3 minutes. Then they were heated to 99° C. for 10 minutes. The samples were then placed on ice for 3 minutes and centrifuged at 13000 rpm for 5 minutes. 100 μl of the top layer in each microfuge tube was then transferred into 96-well plate for PCR later.

PCR: Each PCR reaction mix contained a set of Salmonella enterica-specific primers (forward (SEQ ID NO: 8) and reverse (SEQ ID NO: 9)) (1.0 μM each), a Salmonella enterica specific probe (0.2 μM, SEQ ID NO: 7), and Life Technology Taqman® Universal Mastermix II (1×). The sequence information for primers and probe can be found in Table 1. The same PCR assay conditions from Example 1 was used.

### Results:

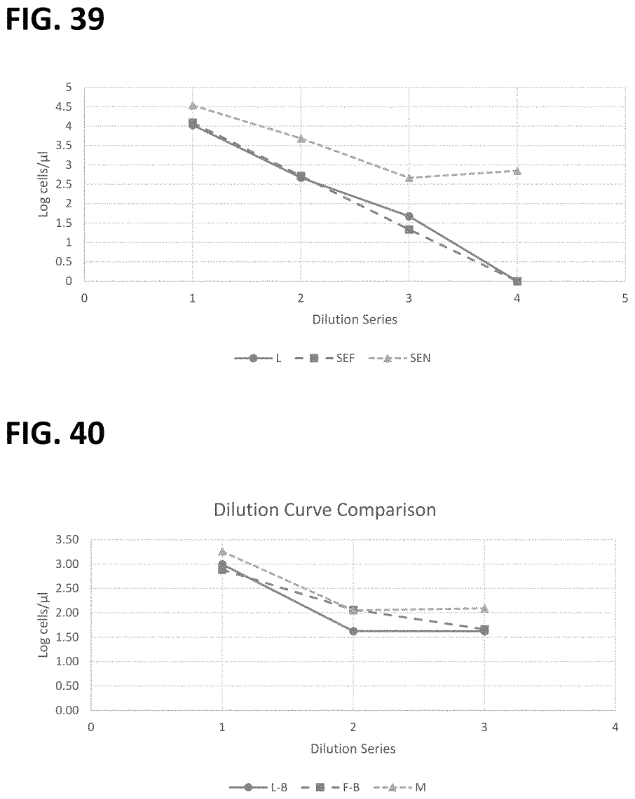

As sample duplicates were used for PCR setup, average PCR data per test sample was obtained to generate the data in FIG. 38 and FIG. 39. The rinsing did not seem to have impact in detecting DNA from live Salmonella in this example according to the data in FIG. 38. This may be due to the filter plate effect or the vacuum effect. The mixed samples (SEN) on FIG. 39 produce higher false positive data than the mixed samples (SEF). SEN samples were not treated with the filtration while SEF samples were treated with the filtration through the 24-well filter plate. There is a significant alignment for the dilution curves of live samples (L) and mixed samples (SEF). This demonstrated the testing efficiency with the 24-well filter plate. This example validates an alternative DNA protocol for Gram-negative bacteria such as Salmonella bacteria after the initial filtration treatment with a 24-well filter plate to isolate live bacterial cells from the free DNA of dead cells.

### Example 17: Viability Testing on Legionella Bacteria

In this example, live cell samples from Legionella pneumophila (LP) were obtained through the filtration method and were used to prepare for three types of test samples: live only samples (L), mixed samples for viability test (F), and mixed samples as process control (M). Two DNA protocols were used to isolate the live cell DNA and mixed cell DNA after the filtration with the 24-well filter plate and the results were compared to determine the efficiency of the viability PCR test. ddPCR was used to analyze viability testing efficiency when low DNA quantity was observed from qPCR data.

### Materials and Methods:

Sample sources: The bacterial suspensions used in this example were from Legionella pneumophila suspension.

Live sample preparation: Seven (7) ml of 1-week-old LP culture was centrifuged at 4500 rpm for 8 minutes. The supernatant was discarded and the pellet was resuspended with 2 ml of sterile H₂O. Then the resuspension was filtered with filter tube via centrifugation to capture live cells. The captured live cells were then resuspended on the filter with H₂O and then transferred to a new tube LPA2 (final volume was 20 ml). 5 ml was set aside for dead LP DNA production. Then, two 10-fold dilutions were made from LPA2 to produce LPB (1/10 dilution of LPA2) and LPC (1/100 dilution of LPA2). About 15 ml was the final volume for LPA2, LPB, and LPC.

Prepare dead LP cell free DNA. Five (5) ml LPA2 was used to create dead LP DNA. Use the filtration method to capture the live cells on the filter. Then appropriate amount of TE and glass beads were used to break down the live cells on the filter. The samples were then centrifuged at 13K rpm for 5 minutes and the filtrate was transferred to a new tube and would be used as dead cell stock. The final volume was brought up to 10 ml.

Test sample preparation: Mixed cell samples were composed of live cell sample and dead cell DNA sample. (e.g., 3000 μl of live sample of different dilutions and 500 μl of the same dead cell stock were combined in each mixed cell sample). Different diluted live cell samples were combined with a fixed amount of dead cell stock to produce a set of mixed samples for this study. Three types of test samples were prepared in this example: L samples: live cell samples at three 10-fold dilutions and labeled as L1, L2, and L3 and placed in the wells of the 24-well filter plate; F samples: mixed cell samples used for viability testing treatment and labeled as F1, F2, and F3 and placed in the wells of the 24-well filter plate; and M samples: mixed cell samples used as a test control without viability testing treatment and labeled as M1, M2, and M3 and placed in 15 ml conical tubes.

Viability test with filter plate treatment. The samples were prepared and placed inside wells of a 24 well filter plate on top of a 24 well plate on the manifold connected with a vacuum pump. The vacuum was turned on to remove liquid inside the filter plate and the filtrate collected in the bottom plate was discarded. 1 ml of H₂O was added to the bottom plate to rinse the sample wells of the bottom plate. The filter plate was placed back on to the top of the bottom plate and 400 μl of TE added to the sample wells of the filter plate. Glass beads were then added to each sample well of the filter plate which was then covered with aluminum foil. The filter plate and the bottom plate were transferred together to a mixer and shaken for 3 minutes. The filter plate complex was then placed back to the device connected with a vacuum pump. The vacuum was turned on to allow liquid to filter through to the bottom plate. The filtrate was obtained and transferred to microfuge tubes. 20 μl of the filtrate was retained for PCR analysis. The remainder was used for DNA extraction with the long protocol (described below).

DNA extraction: As the same sample was used for two different DNA protocols, “—B” was added to the sample name such as L or F to help to identify the DNA samples isolated from the long DNA protocol. For example, F1-B or L1-B, etc. The long protocol described in Example 13 was used for DNA extraction in this example.

PCR: In this example, both qPCR and ddPCR were used to analyze the viability data to gain better understanding of the test results.

qPCR procedure: Each PCR reaction mix contained a set of Legionella pneumophila-specific primers (forward (SEQ ID NO: 2) and reverse (SEQ ID NO: 3), 1.0 μM each), a Legionella pneumophila specific probe (0.2 μM, SEQ ID NO: 1), and Life Technology Taqman® Universal Mastermix II (1×). The sequence information for primers and probe can be found in Table 1. The same PCR assay conditions from Example 1 was used.

ddPCR procedure: Five (5) μl of each sample was combined with 17 μl of the ddPCR reaction mix to bring up to 22 μl per reaction. Each PCR reaction mix contained a set of Target-specific primers (forward (SEQ ID NO: 2) and reverse (SEQ ID NO: 3)) (1.0 μM), a Target specific probe (0.2 μM, SEQ ID NO: 1), and Bio-Rad 2×ddPCR Supermix for probes (No dUTP). Legionella pneumophila-specific primers and probe have been designed and validated at Cicadea Biotech. One replicate per sample were tested by ddPCR. H₂O was used as the negative control. 20 μl of the ddPCR reaction was used for droplet generation and then 40 μl was transferred into a 96-well PCR plate which was placed on the Bio-Rad thermocycler with these cycling conditions: 1 cycle of 95° C. for 10 minutes; then 40 cycles of 94° C. for 30 seconds and 60° C. for 1 minute; 1 cycle of 98° C. for 10 minutes; hold at 4° C. until canceled. The ddPCR data was obtained from a QX200 droplet reader.

### Results:

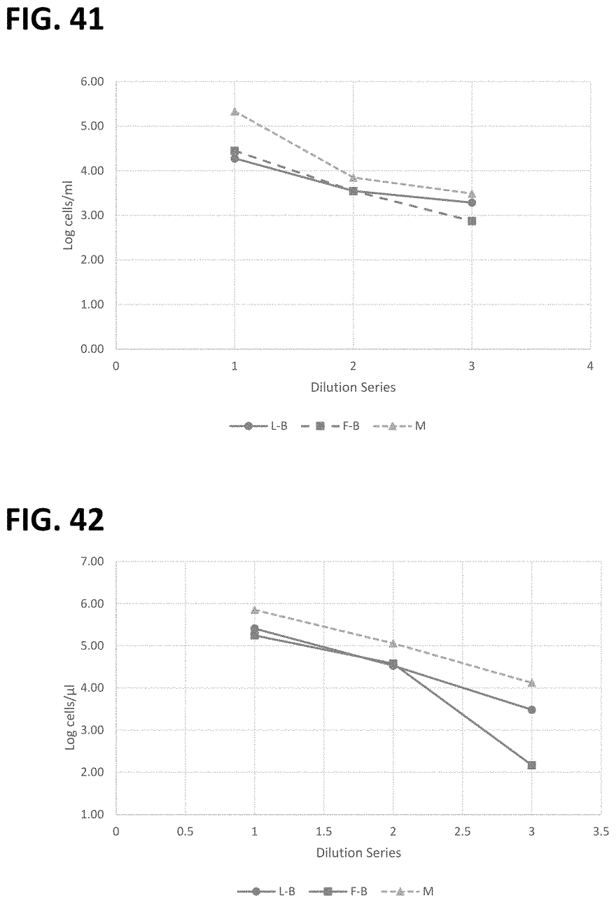

As sample duplicates were used for qPCR setup, average PCR data per test sample was obtained to generate the following data in the tables and the graphs. Only single replicate was used for ddPCR and the ddPCR data is from one data per test sample. In the figures, L-B and F-B are referred to DNA from samples L and F with the long protocol. The qPCR data from FIG. 40 indicated low DNA quantity and no data was found from short protocol. As ddPCR has become a innovative PCR technology to detect low quantity DNA, in these experiments the test samples were run with ddPCR to gain a better understanding on the viability test results in this example. The ddPCR data from FIG. 41 demonstrated the effectiveness of the filtration strategy for viability testing. The unfiltered samples (M) produced higher false positives than the filtered samples (F) and live sample (L). This is the first report on using non-DNA binding filter to isolate DNA from live Legionella bacteria. This testing strategy will play an important role in detecting live Legionella from environmental samples such as different water sources. In addition, the combination of ddPCR to the proposed viability testing strategy added additional benefits in testing accuracy and sensitivity.

### Example 18: Viability Testing on Legionella Bacteria

In this example, live cell samples from Legionella pneumophila (LP) were obtained through the filtration method and were used to prepare for three types of test samples: live only samples (L), mixed samples for viability test (F), and mixed samples as process control (M). Two DNA protocols were used to isolate the live cell DNA and mixed cell DNA after the filtration with the filter tubes and the results were compared to determine the efficiency of the viability PCR test.

### Materials and Methods:

Sample sources: The bacterial suspensions used in this example were from Legionella pneumophila suspension.

Live cell sample preparation: Seven (7) ml of 1-week-old LP culture was centrifuged at 4500 rpm for 8 minutes. The supernatant was discarded and the pellet was resuspended with 2 ml of sterile H₂O. Then the resuspension was filtered with filter tube via centrifugation to capture live cells. The captured live cells were then resuspended on the filter and then transferred to a new tube LPA2 (final volume was 2400 μl). 1 ml was set aside for dead LP DNA production. Then, two 10-fold dilutions were made from LPA2 to produce LPB (1/10 dilution of LPA2) and LPC (1/100 dilution of LPA2). About 1 ml was the final volume for LPA2, LPB, and LPC.

Preparation for dead LP cell free DNA: 1 ml of LPA2 was transferred to a filter tube at two different times to ensure all live LP cells were captured on the filter. Then 500 μl TE and beads were added and the sample tube was shaken for 3 minutes. The sample was then centrifuged at 13K rpm for 3 minutes. The filtrate was transferred to a new tube as dLP. Additional 500 μl TE was added to the filter tube and samples were centrifuged at 13K for 3 minutes. The filtrate was transferred to the tube dLP. 500 μl of H₂O was added to dLP to bring up the final volume of 1500 μl.

Test sample preparation: Mixed cell samples were composed of live cell sample and dead cell DNA sample. (e.g., 200 μl of live sample of different dilutions and 200 μl of the same dead cell stock were combined in each mixed cell sample). Different diluted live cell samples were combined with a fixed amount of dead cell stock to produce a set of mixed samples for this study. Three types of test samples were prepared in this example. L samples: live cell samples at three 10-fold dilutions and they were labeled as L1, L2, and L3. F samples: mixed cell samples used for viability testing treatment and labeled as F1, F2, and F3. M samples: mixed cell samples used as a test control without viability testing treatment and labeled as M1, M2, and M3.

Viability test treatment: Filter tubes were used for viability test in this example. The viability testing process is illustrated as in diagram (FIG. 29). Both L and MF samples were placed in different filter tubes and centrifuged at 13K rpm for 5 minutes. The filtrate containing dead cell DNA in test sample was discarded. The bottom tube was rinsed with sterile H₂O twice. Keep the filter in the same tube and proceed with DNA extraction as follows.

DNA extraction: As the same sample was used for two different DNA protocol, “—B” was added to the sample name such as L or MF to help to identify the DNA samples isolated from the long DNA protocol. For example, L1-B or F1-B, etc. Protocols (long and short) described in Example 13 were used for DNA extraction.

PCR. Each PCR reaction mix contained a set of Legionella pneumophila-specific primers (forward (SEQ ID NO: 2) and reverse (SEQ ID NO: 3)) (1.0 μM each), a Legionella pneumophila specific probe (0.2 μM, SEQ ID NO: 1), and Life Technology Taqman® Universal Mastermix II (1×). The sequence information for primers and probe can be found in Table 1. The same PCR assay conditions from Example 1 was used.

### Results:

As sample duplicates were used for qPCR setup, average PCR data per test sample was obtain to generate the following data in the tables and the graphs. Only single replicate was used for ddPCR and the ddPCR data is from one data per test sample.

The PCR data from FIG. 42 demonstrated the effectiveness of our viability testing strategy. M samples produce higher false positives when comparing with live samples (L) and filtered mixed samples (F). This further confirms that the non-DNA binding filter can be used to isolate DNA from live cells and the DNA can then be used for PCR analysis. This example and Example 17 provide evidence that the proposed technology can be applicable to the detection of many important bacteria such as Legionella pneumophila.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A screening method for detecting the presence of one or more live cells of interest in a test sample suspected of comprising the live cells of interest and at least one dead cell or free nucleic acid material, the method comprising:
(a) obtaining or having obtained a retentate formed by filtering the test sample through a filter, wherein the retentate comprises live cells present in the test sample;
(b) lysing or disrupting the live cells in the retentate to form a lysed/disrupted cell mixture comprising nucleic acid from the lysed/disrupted cells;
(c) filtering the lysed/disrupted cell mixture from (b) through the filter to form a filtrate comprising the nucleic acid from the lysed/disrupted cells;
(d) subjecting the filtrate from (c) to a polymerase chain reaction (PCR) to amplify the nucleic acid from the lysed/disrupted cells;
(e) analyzing the amplified nucleic acid to determine whether the live cells of interest are present in the test sample.

2. The method of claim 1, further comprising filtering the test sample to form the retentate comprising the live cells and a first filtrate comprising free nucleic acid, dead cell material, or a combination thereof.

3. The method of claim 1, wherein the one or more live cells suspected of being present in the test sample comprise one or more microorganisms.

4. The method of claim 1, wherein the one or more live cells suspected of being present in the test sample comprise one or more of bacteria, archaea, fungi, yeasts, molds, algae, protists, or protozoa.

5. The method of claim 1, wherein the filter does not bind or poorly binds free nucleic acids such that a filtrate formed in step (a) comprises a majority of free nucleic acid present in the test sample.

6. The method of claim 1, wherein the filter has pores having a diameter of less than 0.5, less than 0.4, less than 0.3 or less than 0.2 microns.

7. The method of claim 1, wherein the test sample comprises a fluid test sample or a solid or semisolid prepared as a fluid suspension or solution.

8. The method of claim 1, wherein the method is free of an added nucleic acid binding agent for extracting free nucleic acid from the test sample.

9. The method of claim 7, wherein the test sample comprises waste water, household water, municipal water, irrigation water, water obtained from a water tank or water reservoir, swimming pool water, bodies of water, building coolant fluid, building heater fluid, environmental water and fluids, fluid or solids from microbial fermentations, human or animal bodily tissue, solids, or fluid, human waste products and materials, food or drink, animal feed, pet food, medical supplies, swab or survey collections from solid surfaces, medical waste, human and animal drugs, nutraceuticals, probiotic or prebiotic supplements, plant materials, soil, agriculture production inputs, harvested agriculture commodities or produce, gaseous material, or a fluid sample prepared therefrom.

10. The method of claim 8, wherein the test sample comprising food or drink comprises a feed, milk, meat, fermented products, beer, wine, cheese, soy sauce, fish or shellfish, seafood, poultry, fruit, vegetables or eggs or products made therefrom.

11. The method of claim 1, wherein lysing or disrupting the cells comprises agitating the live cells in the presence of beads, granules pellets or other solid material suitable for disrupting cell membranes.

12. The method of claim 11, wherein agitating the live cells comprises sonication, magnetic agitation, use of a forced gas of any combination thereof.

13. The method of claim 1, wherein filtering the test sample and/or filtering the lysed/disrupted cell mixture further comprises applying a centrifugal or vacuum force across the filter.

14. The method of claim 1, wherein the polymerase chain reaction comprises Real-Time PCR (quantitative PCR or qPCR), Reverse-Transcriptase (RT-PCR), Multiplex PCR, Nested PCR, High Fidelity PCR, Fast PCR, Hot Start PCR, Long-range PCR, Arbitrary Primed PCR, Digital PCR, Droplet Digital PCR (ddPCR), isothermal amplification PCR, or Endpoint PCR (Qualitative PCR).

15. The method of claim 1, wherein the polymerase chain reaction comprises qPCR or ddPCR.

16. The method of claim 1, wherein analyzing the amplified nucleic acid comprises sequencing, quantitating, or qualitatively detecting the amplified nucleic acid.

17. The method of claim 16, wherein sequencing the amplified nucleic acid comprises Sanger sequencing, single molecule real time (SMRT) sequencing, nanopore DNA sequencing, massively parallel signature sequencing (MPSS), colony sequencing, 454 pyrosequencing, illumina sequencing, combinatorial probe anchor synthesis (cPAS), SOLiD sequencing, ion torrent semiconductor sequencing, DNA nanoball sequencing, heliscope single molecule sequencing, or using a microfluidic system.

18. The method of claim 16, wherein quantitating the amplified nucleic acid comprises using a nanopore, an oligonucleotide probe, or a nucleic acid dye.

19. The method of claim 16, wherein qualitatively detecting the amplified nucleic acid comprises using an agarose gel, polyacrylamide electrophoresis, restriction endonuclease digestion, dot blots, liquid chromatography, electrochemoluminescence, or a combination of any thereof.

20. The method of claim 3, wherein the live cells suspected of being present in the test sample are pathogenic or toxin producing, or indicative of the presence of pathogenic or toxin producing live cells.

21. The method of claim 3, wherein the live cells suspected of being present in the test sample comprise one or more bacteria, archaea, fungi, yeasts, molds, algae, protists, or protozoa.

22. The method of claim 21, wherein the protist or protozoan is an amoeba, cryptosporidium, or Giardia.

23. The method of claim 3, wherein the microorganism is a mycoplasma, or a bacterium which is a Gram-negative bacterium, or a Gram-positive bacterium.

24. The method of claim 23, wherein the bacterium is selected from any one of the genera Legionella, Escherichia, Salmonella, Listeria, Clostridium, Vibrio, Proteus, Pseudomonas, Borrelia, Mycobacterium, Bacillus, Klebsiella, Yersinia, Enterococcus, Aerococcus, Brucella, Shigella, Acinetobacter, Candidatus, Staphylococcus, Enterococcus, or is a fecal coliform bacterium.

25. A processed test sample prepared from an original test sample for PCR amplification of nucleic acid from live cells of interest suspected of being present in the original test sample, the processed test sample comprising nucleic acid from the live cells substantially free of free nucleic acid and/or dead cells present in the original test sample.

26. The processed test sample of claim 25, wherein the processed test sample is obtained by a process comprising a first filtering of the original test sample through a filter to form a retentate, wherein the retentate comprises a majority of the live cells from the test sample; lysing the live cells in the retentate to form a lysed cell mixture comprising nucleic acid from the lysed cells; and filtering the lysed cell mixture through the filter to form a filtrate comprising the nucleic acid from the lysed cells.

27. The processed test sample of claim 25, free of an added nucleic acid binding agent for extracting free nucleic acid from the test sample.

28. The processed test sample of claim 25, comprising nucleic acid from one or more live microorganisms present in the original sample.

29. The processed test sample of claim 25, wherein the live cells of interest are pathogenic or toxin producing or indicative of the presence of pathogenic or toxin producing live cells.

30. The processed test sample of claim 25, wherein the live cells of interest comprise one or more microorganisms selected from bacteria, archaea, fungi, yeasts, molds, algae, protists, or protozoa.

31. The processed test sample of claim 30, wherein the microorganisms comprise one or more of a mycoplasma, a Gram negative bacterium, or a Gram positive bacterium.

32. The processed test sample of claim 31, wherein the bacteria are selected from the genera Legionella, Escherichia, Salmonella, Listeria, Candidatus, Vibrio, Proteus, Pseudomonas, Borrelia, Mycobacterium, Bacillus, Klebsiella, Yersinia, Enterococcus, Aerococcus, Brucella, Shigella, Acinetobacter, Clostridium, Staphylococcus, Enterococcus, Pseudomonas, Acinetobacter, Streptococcus, Klebsiella, or are fecal coliform bacteria

33. The processed test sample of claim 26, wherein the filter does not bind nucleic acids or poorly binds nucleic acids such that a filtrate formed from filtering the original test sample through the filter comprises a majority of free nucleic acid present in the original test sample.

34. The processed test sample of claim 26, wherein the filter has pores having a diameter of less than 0.5, less than 0.4, less than 0.3 or less than 0.2 microns.

35. The processed test sample of claim 25, wherein the original test sample comprises a fluid test sample or a solid or a semisolid prepared as a fluid suspension or solution.

36. The processed test sample of claim 25, wherein the original test sample comprises waste water, household water, municipal water, irrigation water, water obtained from a water tank or water reservoir, swimming pool water, building coolant fluid, building heater fluid, environmental water and fluids, fluid from microbial fermentations, human or animal bodily fluid, fluid obtained from food or drink, plant materials, soil, gaseous material, or a fluid sample prepared therefrom.

37. The processed test sample of claim 26, wherein lysing the cells comprises agitating the live cells in the presence of beads, granules pellets or other solid material suitable for disrupting cell membranes.

38. The processed test sample of claim 37, wherein agitating the live cells comprises sonication, magnetic agitation, use of a forced gas or any combination thereof.

39. The processed test sample of any of claim 26, wherein filtering the original test sample and/or filtering the lysed cell mixture further comprises applying a centrifugal or vacuum force across the filter.


SEQUENCE LISTING (21 sequences)

SEQ ID NO: 1 - DNA, 20 bp, Artificial Sequence
  synthetic construct - probe
cgccactcatagcgtcttgc

SEQ ID NO: 2 - DNA, 18 bp, Artificial Sequence
  synthetic construct - primer
ggcatagatgttaatccg

SEQ ID NO: 3 - DNA, 18 bp, Artificial Sequence
  synthetic construct - primer
ttcggttaaagccaattg

SEQ ID NO: 4 - DNA, 20 bp, Artificial Sequence
  synthetic construct - probe
tgcaccaataaggccgccaa

SEQ ID NO: 5 - DNA, 18 bp, Artificial Sequence
  synthetic construct - primer
ctggtgatgatacaggaa

SEQ ID NO: 6 - DNA, 21 bp, Artificial Sequence
  synthetic construct - primer
ggttgaacatatttcagtgta

SEQ ID NO: 7 - DNA, 20 bp, Artificial Sequence
  synthetic construct - probe
tcttcctgcgatgcctcaca

SEQ ID NO: 8 - DNA, 18 bp, Artificial Sequence
  synthetic construct - primer
cagccattcctcaagtaa

SEQ ID NO: 9 - DNA, 18 bp, Artificial Sequence
  synthetic construct - primer
ccagcataccgttcttaa

SEQ ID NO: 10 - DNA, 20 bp, Artificial Sequence
  synthetic construct - probe
ccgatccgagcgatggcata

SEQ ID NO: 11 - DNA, 18 bp, Artificial Sequence
  synthetic construct - primer
ggaactggaatggtactc

SEQ ID NO: 12 - DNA, 18 bp, Artificial Sequence
  synthetic construct - primer
ggatcgttggagatagtc

SEQ ID NO: 13 - DNA, 23 bp, Artificial Sequence
  synthetic construct - probe
cgcctgcaagtcctaagacgcca

SEQ ID NO: 14 - DNA, 19 bp, Artificial Sequence
  synthetic construct - primer
tttcatccatggcaccacc

SEQ ID NO: 15 - DNA, 20 bp, Artificial Sequence
  synthetic construct - primer
atccgcgtgtttcttttcga

SEQ ID NO: 16 - DNA, 20 bp, Artificial Sequence
  synthetic construct - probe
ccaattcctctgcccgcttt

SEQ ID NO: 17 - DNA, 24 bp, Artificial Sequence
  synthetic construct - primer
ctggatgatatttactatttatcg

SEQ ID NO: 18 - DNA, 21 bp, Artificial Sequence
  synthetic construct - primer
gtcagagattctttgactaaa

SEQ ID NO: 19 - DNA, 22 bp, Artificial Sequence
  synthetic construct - probe
acagtcatccccgtaatttgcg

SEQ ID NO: 20 - DNA, 19 bp, Artificial Sequence
  synthetic construct - primer
ccaacagatggaatcttca

SEQ ID NO: 21 - DNA, 20 bp, Artificial Sequence
  synthetic construct - primer
ctccattatgacaggcatta